(19) United States
(12) Patent Application Publication
(10) Pub. No.: US 2016/0250298 A1
(43) Pub. Date: Sep. 1, 2016

(54) INCLUSION BODIES FOR TRANSDERMAL DELIVERY OF THERAPEUTIC AND COSMETIC AGENTS

(71) Applicant: SPHERIUM BIOMED S.L., Barcelona (ES)

(72) Inventors: Luis RUIZ-AVILA, Barcelona (ES); Ramon BOSSER ARTAL, Barcelona (ES)

(21) Appl. No.: 15/033,492

(22) PCT Filed: Oct. 31, 2014
(86) PCT No.: PCT/IB2014/003004

§ 371 Date: Apr. 29, 2016

Related U.S. Application Data
(60) Provisional application No. 61/898,698, filed on Nov. 1, 2013.

(51) Int. Cl. A61K 38/44; A61K 9/00; A61K 8/64; A61K 8/66; A61K 38/20; A61K 8/02; A61Q 19/06; A61Q 19/08; A61Q 5/00; A61Q 19/00; A61K 9/14; A61K 38/17; A61K 38/18

(52) U.S. Cl. CPC A61K 38/44; A61K 38/1767; A61K 9/0014; A61K 8/64; A61K 38/17; A61K 8/66; A61K 38/2066; A61K 38/1808; A61K 38/18; A61K 38/1866; A61Q 19/06; A61Q 19/08; A61Q 5/006; A61Q 19/007; A61K 9/14; A61K 8/0241; C12Y 111/01006; A61K 2800/413

(57) ABSTRACT

The present invention relates to methods, compositions, and devices for the transdermal delivery of therapeutic agents and cosmetic agents in inclusion body form. Such inclusion bodies can deliver therapeutic and cosmetic agents through the skin barrier and reach locations deep in the skin. The disclosed compositions can be used to treat skins diseases and conditions.

### BACKGROUND

The present invention provides methods and compositions for transdermal delivery of cosmetic and therapeutic agents in inclusion body form.

The skin provides a protective barrier against foreign materials and infection. In mammals this is mainly accomplished by forming a highly insoluble protein and lipid structure on the surface of the skin termed the stratum corneum (SC) (Downing et al., Dermatology in General Medicine, Fitzpatrick, et al., eds., pp. 210-221 (1993), Ponec, M., The Keratinocyte Handbook, Leigh, et al., eds., pp. 351-363 (1994)). The stratum corneum forms a barrier (sometimes referred to as the skin barrier) that protects underlying tissue from infection, dehydration, chemicals, and mechanical stress. Cells of the stratum corneum contain a dense network of keratin, a protein that helps keep the skin hydrated by preventing water evaporation. These cells can also absorb water, further aiding in hydration. In addition, this layer is responsible for the “spring back” or elastic properties of skin. The thickness of the stratum corneum varies throughout the body. In the palms of the hands and the soles of the feet (sometimes knees, elbows, knuckles, and elsewhere) this layer is stabilized and built by the stratum lucidum (clear phase) which allows the cells to concentrate keratin and toughen them before they rise into a typically thicker, more cohesive SC. In general, the stratum corneum contains 15 to 20 layers of dead cells. The stratum corneum has a thickness between 10 and 40 μm.

Because of the accessibility and large area of the skin, it has long been considered a promising route for the administration of drugs, whether dermal, regional, or systemic effects are desired. A topical route of drug administration is sometimes desirable because the risks and inconvenience of parenteral treatment can be avoided. Also, the variable absorption and metabolism associated with oral treatment can be circumvented, and drug administration can be continuous, thereby permitting the use of therapeutically active agents with short biological half-lives. An additional advantage is that the gastrointestinal irritation associated with many compounds can be avoided; and cutaneous manifestations of diseases can be treated more effectively than by systemic approaches. However, epidermal penetration of high molecular weight therapeutic or cosmetic agents, in particular proteins and peptides, is in the vast majority of cases substantially limited.

Most transdermal delivery compositions (e.g., therapeutic or cosmetic compositions) achieve epidermal penetration by using a skin penetration enhancing carrier or vehicle. Such carrier or vehicles (which are compounds or mixtures of compounds) are known in the art as “penetration enhancers” or “skin enhancers.” While some skin enhancers in the literature enhance transdermal absorption, they possess certain drawbacks in that (i) some are regarded as toxic; (ii) some irritate the skin; (iii) some have a thinning effect on the skin after prolonged use; (iv) some change the intactness of the skin structure resulting in a change in the diffusability of the drug; and (v) all are incapable of delivering high molecular weight pharmaceuticals and cosmetic agents, for example, peptides and proteins.

Another alternative approach is to locally damage the skin via micropunctures or abrasions to facilitate the penetration of the proteins or peptides. Again, these methods irritate and damage the skin and can lead to long term skin damage. Accordingly, there remains a need for the development of transdermal delivery compositions and methods capable of delivering a wide-range of pharmaceuticals and cosmetic agents, in particular high molecular weight molecules (e.g., proteins and peptides), through the skin barrier.

### BRIEF SUMMARY

The present disclosure provides a method of delivering a cosmetic agent across the skin barrier comprising applying to the skin of a subject a cosmetic composition comprising at least one cosmetic agent in inclusion body form, wherein the at least one cosmetic agent crosses the skin barrier in inclusion body form. Also provided is a method of delivering a therapeutic agent across the skin barrier comprising applying to the skin of a subject in need thereof a therapeutic composition comprising at least one therapeutic agent in inclusion body form, wherein the at least one therapeutic agent crosses the skin barrier in inclusion body form.

In addition the instant disclosure provides a method for treating a skin condition in a subject in need thereof comprising topically applying a cosmetically effective amount of a cosmetic composition comprising at least one cosmetic agent in inclusion body form, and a dermatologically acceptable carrier to the skin of the subject so as to improve the skin condition of the subject.

The present disclosure also provides a method for treating a skin condition in a subject in need thereof comprising topically applying a therapeutically effective amount of a therapeutic composition comprising at least one therapeutic agent in inclusion body form, and a pharmaceutically acceptable carrier to the skin of the subject so as to improve the skin condition of the subject. Also provided is a method of enhancing penetration of the skin by a cosmetic agent comprising applying to the skin of a subject a cosmetic composition comprising at least one cosmetic agent in inclusion body form, wherein the penetration of the cosmetic agent is increased with respect to the penetration of the same cosmetic agent in soluble form.

Also provided is a method of enhancing penetration of the skin by a therapeutic agent comprising applying to the skin of a subject in need thereof a therapeutic composition comprising at least one therapeutic agent in inclusion body form, wherein the penetration of the therapeutic agent is increased with respect to the penetration of the same therapeutic agent in soluble form.

The disclosure also provides a method of stimulating tissue regeneration, comprising applying to the skin of a subject in need thereof at least one cosmetic agent or therapeutic agent in inclusion body form, wherein the inclusion body penetrates the skin barrier and reaches said tissue and stimulates its regeneration. Also provided is a method of stimulating eukaryotic cell proliferation, comprising applying to the skin of a subject in need thereof at least at least one cosmetic agent or therapeutic agent in isolated inclusion body form, wherein the inclusion body penetrates the skin barrier and stimulates eukaryotic cell proliferation. Also provided is a method of making a transdermal delivery system comprising (i) providing at least one cosmetic agent or a therapeutic agent in inclusion body form, (ii) mixing the inclusion body with a carrier, and (iii) mixing the inclusion body with the carrier, thereby making the transdermal delivery system.

In some aspects of the methods and compositions disclosed herein, the inclusion body is insoluble. In other aspects, the inclusion body is not solubilized. In some aspects, the inclusion body is partially solubilized. In other aspects, the inclusion body is in particulate form. In some aspects, the particulate form has a particle size between about 20 nm and about 1500 nm. In some aspects, the particulate form has a particle size between about 100 nm and about 500 nm. In other aspects, the particulate form has a particle size between about 150 nm and about 300 nm.

In some aspects, the particulate form is in hydrated amorphous form. In some aspects, the inclusion body is internalized by a target cell. In some aspects, the target cell is an epidermal cell. In other aspects, the target cell is a non-epidermal cell. In some aspects, the target cell is a neuron. In other aspects, the target cell is a muscle cell. In some aspects, the target cell is an adipocyte.

In some aspects, the inclusion body can penetrate at least one skin layer. In other aspects, the inclusion body can penetrate the cornified layer (stratum corneum), translucent layer (stratum lucidum), granular layer (stratum granulosum), spinous layer (stratum spinosum) or basal/germinal layer (stratum basale/germinativum).

In some aspects of the methods and compositions disclosed herein, the cosmetic agent or therapeutic agent comprises a polypeptide. In some aspects, the polypeptide is biologically active. In other aspects, the polypeptide is a prodrug. In some aspects, the polypeptide is a recombinant polypeptide or a fragment thereof, a natural polypeptide or a fragment thereof, or a chemically synthesized polypeptide. In some aspects, the polypeptide is a fusion protein. In other aspects, the polypeptide is a protein conjugate. In some aspects, the polypeptide is chimeric. In other aspects, the recombinant polypeptide is expressed in a cell selected from the group consisting of bacteria, yeasts, insect cells, and mammalian cells.

In some aspects of the methods and compositions disclosed herein, the polypeptide is genetically fused or conjugated to an inclusion-body inducing polypeptide. In some aspects, the inclusion-body inducing polypeptide is a viral protein. In some aspects, the viral protein is a capsid protein. In some aspects, the inclusion body-inducing polypeptide comprises the VP1 pentamer-forming capsid protein of Foot and Mouth Disease Virus (FMDV) or a fragment thereof. In some aspects, the polypeptide is conjugated to a protein purification tag or a visualization tag. In some aspects, the protein purification tag is a His6-tag. In other aspects, the visualization tag is a fluorescent tag.

In some aspects of the methods and compositions disclosed herein, the polypeptide is selected from the group consisting of erythropoietin (EPO), corticotropin-releasing hormone (CRH), growth hormone-releasing hormone (GHRH), gonadotropin-releasing hormone (GnRH), thyrotropin-releasing hormone (TRH), prolactin-releasing hormone (PRH), melanotropin-releasing hormone (MRH), prolactin-inhibiting hormone (PIH), somatostatin, adrenocorticotropic hormone (ACTH), somatotropin or growth hormone (GH), luteinizing hormone (LH), follicle-stimulating hormone (FSH), thyrotropin (TSH or thyroid-stimulating hormone), prolactin, oxytocin, antidiuretic hormone (ADH or vasopressin), melatonin, Müllerian inhibiting factor, calcitonin, parathyroid hormone, gastrin, cholecystokinin (CCK), secretin, insulin-like growth factor type I (IGF-I), insulin-like growth factor type II (IGF-II), atrial natriuretic peptide (ANP), human chorionic gonadotropin (hCG), insulin, glucagon, somatostatin, pancreatic polypeptide (PP), leptin, neuropeptide Y, renin, angiotensin I, angiotensin II, factor VIII, factor IX, tissue factor, factor VII, factor X, thrombin, factor V, factor XI, factor XIII, interleukin 1 (IL-1), Tumor Necrosis Factor Alpha (TNF-α), interleukin 6 (IL-6), interleukin 8 (IL-8), interleukin-10 (IL-10), interleukin 12 (IL-12), interleukin 16 (IL-16), interferons alpha, beta, gamma, nerve growth factor (NGF), platelet-derived growth factor (PDGF), transforming growth factor beta (TGF-beta), bone morphogenetic proteins (BMPs), fibroblast growth factor-(FGF), epidermal growth factor (EGF), vascular endothelial growth factor (VEGF), granulocyte colony-stimulating factor (G-CSF), glial growth factor, keratinocyte growth factor (KGF), endothelial growth factor, alpha-1 antitrypsin, granulocyte-macrophage colony-stimulating factor (GM-CSF), cyclosporine, fibrinogen, lactoferrin, tissue-type plasminogen activator (tPA), chymotrypsin, immunoglobins, hirudin, superoxide dismutase, imighicerase, dihydrofolate reductase (DHFR), catalase, or chaperones. In some aspects, the polypeptide comprises Hsp70 or a functional fragment thereof. In other aspects, the polypeptide comprises, consists, or consists essentially of IL-1U and/or EGF and/or KGF and/or VEGF, and/or fragments, variants, or derivatives thereof.

In some aspects of the methods and compositions disclosed herein, the skin condition to be treated with a cosmetic agent is selected from psoriasis, cellulite, acne vulgaris, acne cystic, skin aging, skin wrinkles, hyperpigmentation, keratosis, skin blemish, dandruff, warts, photodamaged skin, chronic dermatoses, dermatitis, dryness, ichthyosis, viral infections, fungal infections, and bacterial skin infections. In some aspects, the skin condition to be treated with a therapeutic agent is selected from psoriasis, acne, athlete's foot, canker sore, carbuncle, candidiasis, bacterial vaginitis, vaginosis, cellulitis, cold sores, dandruff, dermatitis, eczema, erythrasma, erysipelas, erythema multiforme, furuncle, impetigo, and infection.

The present disclosure also provides a transdermal delivery system comprising a cosmetic composition comprising at least one cosmetic agent in inclusion body form. Also provided is a transdermal delivery system comprising a therapeutic composition comprising at least one therapeutic agent in inclusion body form. In some aspects, the delivery system is a patch, a spray, a swab, a sponge, a stick, or a shampoo.

Also provided is an apparatus comprising a vessel joined to an applicator and a transdermal delivery system disclosed herein incorporated into the vessel.

Also provided is a topical cosmetic composition comprising at least one cosmetic agent in isolated inclusion body form, wherein said inclusion body can penetrate the skin barrier. Also provided is a topical therapeutic composition comprising at least one therapeutic agent in isolated inclusion body form, wherein said inclusion body can penetrate the skin barrier. In some aspects, the composition is a solution, a gel, a cream, a lotion, an ointment, an emulsion, a suspension, an aerosol, an aerosol foam, a liniment, a tincture, a salve, a poultice, a dry power, or a combination thereof.

### BRIEF DESCRIPTION OF THE DRAWINGS/FIGURES

FIG. 1 shows fluorescence microscopy images corresponding to three inserts of reconstituted human skin (STRATICELL® RHE/EPI) in incubation solution. Panel A shows a control, Panel B shown a sample incubated with inclusion bodies (CI), and Panel C shows a sample incubated with soluble GFP.

FIG. 2 shows fluorescence microscopy images corresponding to three samples: control (CTRL) (Panel A), soluble GFP (GFP) (Panel B), and GFP inclusion bodies (CI) (Panel C) after incubation with reconstituted human skin (STRATICELL® RHE/EPI) samples. Magnification and exposure time are indicated for each sample. Panel D, E, and F correspond to transmission microscopy images of the samples in panels A, B, and C, respectively. Panels D, E, and F also show the location of the membrane substrate used to grow the model epidermis (“m”) and the location of the stratum corneum (SC).

FIG. 3 shows a fluorescence microscopy image of GFP inclusion body samples (CI) after incubation with reconstituted human skin (STRATICELL® RHE/EPI) samples.

FIG. 4 shows fluorescence microscopy (panel A) and transmitted light (panel B) images of GFP inclusion body samples (CI) after incubation with reconstituted human skin (STRATICELL® RHE/EPI) samples, showing that some of the stratum corneum (SC) partially detached.

FIG. 5 shows a composite image generated from 3 fluorescence microscopy images of the same field taken at different focal plains (Panel A), and the same image of panel A superimposed to the transmission microscopy image of the sample (Panel B). Model reconstituted human skin (STRATICELL® RHE/EPI) samples were incubated with GFP inclusion bodies.

FIG. 6 shown fluorescence microscopy images of model reconstituted human skin (STRATICELL® RHE/EPI) samples incubated with soluble GFP. Panels A and B show the same field captured with a 4 second exposure time (Panel A) or a 30 second exposure time (Panel B).

FIG. 7 shown fluorescence microscopy images of model reconstituted human skin (STRATICELL® RHE/EPI) control samples. Panels A and B show the same field captured with a 4 second exposure time (Panel A), or a 30 second exposure time (Panel B).

FIG. 8 shows fluorescence images of M0037-CI (inclusion bodies) (Panel A) and M00037-GFP (soluble GFP) (Panel B) incubated with model reconstituted human skin (STRATICELL® RHE/EPI) samples.

FIG. 9 shows that the fluorescent signal from M0037-CI samples (inclusion bodies) was detected as fluorescent aggregates located in the stratum corneum (SC), in the most external layer of the epidermis, and in many cases also in intermediate and deep areas of the epidermis. Show are a confocal fluorescence microscopy image (Panel A), a transmission microscopy image (Panel B), and the confocal fluorescence microscopy image superimposed to the transmission microscopy image (Panel C).

FIG. 10 shows that the majority of the fluorescence of the M0037-GFP samples (soluble GFP) was disperse and located exclusively in the stratum corneum area. The figure shows a confocal fluorescence microscopy image (Panel A), a transmission microscopy image (Panel B), and the confocal fluorescence microscopy image superimposed onto the transmission microscopy image (Panel C).

FIG. 11 shows a diagram of the structure of human epidermis (Panel A), a micrograph showing the structure of human epidermis (Panel B), and an exemplary micrograph of the STRATICELL® Reconstituted Human Epidermis model system (Panel C).

FIG. 12 shows a SDS-PAGE gel stained with Coomassie blue (Panel A) and a Western blot (Panel B) corresponding to the expression of VEGF in L. lactis. Lane 1 corresponds to the Blue Plus2 standard. Lanes 2-7 are soluble extracts. Lanes 8-13 are insoluble extracts. Lane 2: Strain UP1421 (HCN, wt); lane 3: Strain UP1424 (MCN, wt); lane 4: Strain UP1427 (HCN, htrA); lane 5: Strain UP1430 (MCN, htrA); lane 6: Strain UP1433 (IICN, clpP); lane 7: Strain UP1436 (MCN, clpP); lane 8: Strain UP1421 (HCN, wt); lane 9: Strain UP1424 (MCN, wt); lane 10: Strain UP1427 (HCN, htrA); lane 11: Strain UP1430 (MCN, htrA); lane 12: Strain UP1433 (HCN, clpP); lane 13: Strain UP1436 (MCN, clpP).

FIG. 13 shows a SDS-PAGE gel stained with Coomassie blue (Panel A) and Western blot (Panel B) corresponding to the expression of KGF in L. lactis. Lane 1 corresponds to the Blue Plus2 standard. Lanes 2-7 are soluble extracts. Lanes 8-13 are insoluble extracts. Lane 2: Strain UP1422 (HCN, wt); lane 3: Strain UP1425 (MCN, wt); lane 4: Strain UP1428 (HCN, htrA); lane 5: Strain UP1431 (MCN, htrA); lane 6: Strain UP1434 (HCN, clpP); lane 7: Strain UP1437 (MCN, clpP): lane 8: Strain UP1422 (HCN, wt); lane 9: Strain UP1425 (MCN, wt); lane 10: Strain UP1428 (HCN, htrA); lane 11: Strain UP1431 (MCN, htrA); lane 12: Strain UP1434 (HCN, clpP); lane 13: Strain UP1437 (MCN, clpP).

### DETAILED DESCRIPTION

The present disclosure provides methods, compositions, systems, and devices for the transdermal delivery of compositions comprising low, medium, and high molecular weight therapeutic or cosmetic agents (e.g., peptides and proteins). The disclosure includes transdermal delivery compositions with therapeutic and cosmetic application, transdermal delivery devices for providing such transdermal delivery compositions to subjects in need thereof, and methods of making and using the foregoing. In some embodiments, the transdermal delivery compositions disclosed herein can also be used for diagnostics.

### I. Definitions

Before describing the present invention in detail, it is to be understood that this invention is not limited to specific compositions or process steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B.” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. The headings provided herein are not limitations of the various aspects, which can be had by reference to the specification as a whole. Accordingly, the terms defined immediately below are more fully defined by reference to the specification in its entirety.

It is understood that wherever aspects are described herein with the language “comprising,” otherwise analogous aspects described in terms of “consisting of” and/or “consisting essentially of” are also provided.

As used herein, the term “about” typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges.

A polypeptide, protein, inclusion body, or other composition disclosed herein which is “isolated” is a polypeptide, protein, inclusion body, or other composition disclosed herein which is in a form not found in nature. Isolated polypeptide, protein, inclusion body, or other compositions disclosed herein include those which have been purified to a degree that they are no longer in a form in which they are found in nature. In some aspects, a polypeptide, protein, inclusion body, or other composition disclosed herein as isolated is substantially pure.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA. In some aspects, a polynucleotide (e.g., a synthetic polynucleotide) encoding a therapeutic, prophylactic, or cosmetic protein disclosed herein (e.g., a DNA or an RNA) has been codon optimized. Numerous codon optimization methods are known in the art, for example as described in Gould et al. (2014) Front Bioeng. Biotechnol. 2:21; Mauro & Chappell (2014) Trends Mol. Biol pii: S1471-4914(14)00140-3; or Elena et al. (2014) Front. Microbiol. 5:21, all of which are herein incorporated by reference in their entireties.

The term “vector” means a construct, which is capable of delivering, and in some aspects, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. In some aspects, therapeutic, prophylactic, or cosmetic protein disclosed herein can be encoded by more than one nucleic acid, which in turn can be in one or more vectors. Accordingly, when a therapeutic, prophylactic, or cosmetic protein disclosed herein comprises two, three, or more subunits, each of those subunits could be encoded by a polynucleotide. Such polynucleotides could all the inserted in a single vector, under the control of a single promoter or under the control of multiple promoters (e.g., a promoter for each nucleic acid sequence encoding a subunit). In another aspect, each one of the polynucleotides can be inserted in a different vector. If more than one vector is used, the vector could be the same, or a different vector could be used for each polynucleotide.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component (e.g., a dye), a therapeutic agent (e.g., an anticancer agent), or a cosmetic agent. Also included within the definition are, for example, polypeptides containing one or more analogs of an amino acid (including, for example, unnatural amino acids, etc.), as well as other modifications known in the art.

A “recombinant” polypeptide or protein refers to a polypeptide or protein produced via recombinant DNA technology. Recombinantly produced polypeptides and proteins expressed in engineered host cells are considered isolated for the purpose of the invention, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized. Production of recombinant therapeutic agent and cosmetic agents is disclosed more in detail below. The recombinant protein can be, for example:
(a) a native protein;
(b) a mutant protein (e.g., a point mutant or deletion/insertion mutant);
(c) a variant protein (e.g., a protein in which an amino acid normally occurring at a certain position in the native protein is naturally replaced by an alternative amino acid in some natural subpopulations);
(d) a splice variant;
(e) a fragment from a native, mutant, variant, or splice variant protein (e.g., a fragment obtained using recombinant techniques);
(f) a fusion protein comprising two or more therapeutic, prophylactic, or cosmetic protein moieties;
(g) a fusion protein comprising one, two, three or more therapeutic, prophylactic, or cosmetic protein moieties and further comprising at least an additional moiety capable of improving a pharmacokinetic property, e.g., a peptide moiety capable to extending serum half-like such an Fc moiety, albumin, XTEN, HAP, etc. (see, e.g., Hwang et al. (2014) FEBS Letters 588:247-252);
(h) a fusion protein comprising one, two, three or more therapeutic, prophylactic, or cosmetic protein moieties and further comprising at least an detectable moiety for diagnostic methods, e.g., an affinity tag, a fluorescent protein, etc. (see, e.g., Nahalka et al. (2007) Biotechnol. Bioeng. 97:454-461);
(i) a conjugated protein (see, e.g., Talafova et al. (2013) Microbial Cell Factories 12:16; Shiber et al. (2013) Mol. Biol. Cell. 24: 2076-2087); or,
(j) a combination thereof.

The term recombinant protein also encompasses polypeptide comprising non-protein moieties, e.g., carbohydrates, lipids, lipopolysaccharides, nucleic acids, other biochemical entities, or combinations thereof.

As used herein, the term “vaccine immunogen” refers to a protein (e.g., a recombinant protein described above) that elicits a humoral immune response when injected into an animal and comprises, for example, B cell epitopes and T cell epitopes, which is specifically used to prepare a vaccine. While the majority of heterologous proteins can trigger an immune reaction in an organism, the terms immunogen, vaccine immunogen, and grammatical variants thereof refer in the context of the present disclosure to proteins that (i) can elicit an immune response in a subject, and (ii) are used in a vaccine or vaccine-related composition (e.g., a booster preparation to be co-administered with a vaccine). The term “vaccine” is used to define an antigenic preparation used to produce active immunity to a disease, in order to prevent or ameliorate the effects of infection.

As used herein, the expression “inclusion body” or “IB” refers to partial or complete deposits of a recombinant protein(s) in the form of insoluble aggregates which are produced in a microorganism (e.g., a prokaryotic or eukaryotic organism) or in a suitable recombinant expression system (e.g., a cell system or a cell-free system). The abbreviation “IB” will generally be used to refer to an inclusion body (singular) and the abbreviation “IBs” to refer to inclusion bodies (plural). IBs normally have a particle size comprised between 0.05 m and 1 μm, although said range can vary. In some aspects, the IB particle size (measured, e.g., as average diameter size, size of the largest dimension of the IB, or grid size of a sieve that does not allow more than a certain percentage of IBs to go through, e.g., 5%-20%) can be about 0.05 μm, about 0.06 μm, about 0.07 μm, about 0.08 μm, about 0.09 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm or about 20 μm.

In some aspects, the IB particle size is between 20 and 1500 nm. In some aspects the IB particle side is about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1000 μm, about 1100 μm, about 1200 μm, about 1300 μm, about 1400 μm, or about 1500 μm. In some aspects, the IB particle size is larger than 1500 μm.

In some cases, but not all cases, these IBs may be recognized as bright refractive spots under an optical microscope. An IB is normally formed by the aggregation of an insoluble form of the product of a foreign gene. The foreign gene which is introduced into a plasmid could be a gene encoding a heterologous or homologous protein. Heterologous proteins are more likely to form IBs, since they are foreign proteins for the cell generating them. Nevertheless, homologous proteins can also produce IBs, for example, due to fusion to specific sequences which increase the production of the protein in inclusion body form. The overexpressed protein can be a therapeutic agent (for example, a protein or peptide) or a cosmetic agent (for example, a protein or peptide).

The term “inclusion body form” as used herein refers to a cosmetic agent or a therapeutic agent, generally a protein, which is included in an insoluble protein aggregate. In some aspects, a non-protein cosmetic or therapeutic agent (e.g., a nucleic acid) can be incorporated into an inclusion body to yield a topically deliverable particle. In that case, for example, such therapeutic or cosmetic agent (e.g., a nucleic acid) would be considered to be in “inclusion body form.” Administration of a therapeutic agent or cosmetic agent in inclusion body form as disclosed herein also means that the inclusion body can penetrate the skin barrier while maintaining (totally or partially) its integrity as an inclusion body.

“Heterologous proteins” are those proteins foreign to the host cell being utilized, for example, a human protein recombinantly produced by E. coli. While the heterologous protein may be prokaryotic or eukaryotic, preferably it is eukaryotic, more preferably mammalian, and most preferably human. In certain aspects, the heterologous protein is recombinantly produced (e.g., it is a recombinant polypeptide or a recombinant protein).

IBs can accumulate in the cytoplasm or in the periplasm of prokaryotic cells, depending on whether the recombinant protein has been designed to accumulate in the cytoplasm or to be secreted to the periplasm. A therapeutic protein may be directed to the periplasm of a prokaryotic cell or to a certain cellular compartment in the case of an eukaryotic cell wherein said protein would form inclusion bodies.

Protein location may be directed by using a signal sequence. Virtually any signal sequence can be used to put the present invention into practice (e.g., Galliciotti et al. (2001) J. Membrane Biology 183:175-182; Stampolidis et al. (2009) Arch. Biochem. Biophys. 484:8-15; Mergulhio & Monteiro (2007) Methods Mol. Biol. 390:47-61). For example, expressed proteins may be redirected to peroxisomes with a PTS (peroxisomal targeting sequence), to the mitochondrial matrix with a MTS (mitochondrial targeting signal), to the nucleus with a NLS (nuclear localization signal), or to the endoplasmic reticulum with a SRP (signal recognition peptide). Within the context of the present disclosure, the term “signal peptide” includes targeting signals, signal sequences, transit peptides and localization signals.

The recombinant nucleic acid sequence encoding the overexpressed protein may include an inclusion body fusion partner (i.e., inclusion body-inducing protein, peptide or polypeptide) that is operably linked to the therapeutic protein, e.g. the VP1 protein of the foot and mouth disease virus (FMDV), the F19D mutant of human Ab-amyloid protein, or baculoviral polyhedrin (see, e.g., Li et al. (2007) Biotechnol. Bioeng. 96:1183-1190). It is known in the art that linking an inclusion body fusion partner to a preselected polypeptide will cause the tandem polypeptide to form an inclusion body. It is also known in the art that the amino acid sequence of an inclusion body fusion partner can be altered to produce inclusion bodies that exhibit useful characteristics.

The overexpressed protein can typically represent 70-100% of the IB material, which can contain, in small amounts, other proteins (e.g., membrane proteins, etc.), ribosomal components, and a small amount of phospholipids and nucleic acids which are adsorbed after cell lysis. Some chaperones or folding modulators (such as DnaK, GroEL and IbpA/B) are sometimes, but not always, associated with IB formation. In some aspects, overexpressed protein represents at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% of the IB material.

In some aspects, the inclusion bodies disclosed herein comprise interleukin-10 (Il-10), epidermal growth factor (EGF), keratinocyte growth factor (KGF), vascular endothelial growth factor (VEGF), or combinations thereof.

As used herein, the term “VEGF” refers to a sub-family of growth factors, more particularly, the platelet-derived growth factor family of cystine-knot growth factors. The VEGF family includes important signaling proteins involved in both vasculogenesis (the dc novo formation of the embryonic circulatory system) and angiogenesis (the growth of blood vessels from pre-existing vasculature). VEGF family members include VEGF-A (associated with generic angiogenesis), VEGF-B (associated with embryonic angiogenesis), VEGF-C (associated with lymphangiogenesis), VEGF-D (required for the development of lymphatic vasculature surrounding lung bronchioles), and P1GF (important for vasculogenesis, associated with angiogenesis during ischemia, inflammation, wound healing, and cancer).

All members of the VEGF family stimulate cellular responses by binding to tyrosine kinase receptors (the VEGFRs) on the cell surface, causing them to dimerize and become activated through transphosphorylation, although to different sites, times and extents. All VEGF receptors have an extracellular portion consisting of 7 immunoglobulin-like domains, a single transmembrane spanning region, and an intracellular portion containing a split tyrosine-kinase domain. VEGF-A binds to VEGFR-1 (Flt-1) and VEGFR-2 (DR Flk-1). VEGF-C and VEGF-D are ligands for a third receptor (VEGFR-3), which mediates lymphangiogenesis.

As used herein, the term “human VEGF” refers to the 165-amino acid human vascular endothelial cell growth factor, and related human 121-, 189-, and 206-amino acid vascular endothelial cell growth factors, as described by Leung et al, Science 246: 1306 (1989), and Houck et al, Mol. Endocrin. 5:1806 (1991) together with the naturally occurring allelic and processed forms of those growth factors.

The term “VEGF” is also used to refer to truncated forms of the VEGF polypeptide comprising amino acids 8 to 109 or 1 to 109 of the 165-amino acid human vascular endothelial cell growth factor. Reference to any such forms of VEGF may be identified in the present application, e.g., by “VEGF (8-109),” “VEGF (1-109)” or “VEGF165.” The amino acid positions for a truncated native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF. See, for example, Int'l Publ. Nos. WO1997020925, WO2007044534, WO2008116111, WO2005011722, WO2001074317, WO2006138468; European Pat. No. EP1692158B1; or European Publ. No. EP2447280, all of which are herein incorporated by reference in their entireties. The amino acid sequence of VEGF-A is available at the Uniprot database under entry number P15692, including also the sequences of the variants VEGF206 (Uniprot: P15692-1), VEGF189 (Uniprot: P15692-2), VEGF183 (Uniprot: P15692-3), VEGF165 (Uniprot: P15692-4), VEGF148 (Uniprot:P15692-5), VEGF145 (Uniprot: P15692-6), VEGF165B (Uniprot: P15692-8), VEGF121 (Uniprot: P15692-9), VEGF111 (Uniprot: P15692-10), L-VEGF165 (Uniprot: P15692-11), L-VEGF121 (Uniprot: P15692-12), L-VEGF189 (Uniprot: P15692-13), L-VEGF206 (Uniprot: P15692-14), VEGF 15 (Uniprot: P15692-15), VEGF 16 (Uniprot: P15692-16), VEGF 17 (Uniprot: P15692-17), VEGF 18 (Uniprot: P15692-18). The term VEGF includes also non-human forms of the protein.

VEGF assists in stimulating new blood vessel formation by helping to regulate angiogenesis. Blood vessel formation is critical in repairing wounds or damaged skin, accordingly, VEGF administration can be used the treatment of wounds or to treat damaged skin. VEGF is also helpful in increasing blood vessel permeability, thereby enhancing the penetration of other topicals. VEGF administration can be used to reduce the formation of broken capillaries, thus, it can be used to treat conditions such as rosacea.

As used herein, “interleukin-10” or “IL-10” is defined as a protein which (i) has an amino acid sequence of mature IL-10 (e.g., lacking a secretory leader sequence) as disclosed in U.S. Pat. No. 5,231,012 and (ii) has biological activity that is common to native IL-10. Also included are muteins and other analogs, including the Epstein-Barr Virus protein BCRF1 (viral IL-10), which retain the biological activity of IL-10. See, for example, U.S. Pat. No. 5,753,218, U.S. Pat. No. 5,776,451, U.S. Pat. No. 6,544,504, U.S. Pat. No. 7,052,686, U.S. Pat. No. 5,665,345; U.S. Publ. No. US20050158760; or Intl. Publ. No. WO2013130913, WO1997005896, WO2014023673, all of which are herein incorporated by reference in their entireties. The protein sequence of interleukin-10 can be found at the Uniprot database under accession number P22301. The term interleukin-10 encompasses also non-human forms of the protein.

As used herein, the term “keratinocyte growth factor” or “KGF” refers to a member of a group of the FGF family of proteins which is capable of binding to FGFR-2, lacks significant activity on fibroblasts, is uniquely specific for epithelial cells and is particularly active on keratinocytes. KGF, analogs and fragments thereof may be synthetically or recombinantly produced. Moreover, KGF may be isolated from natural sources, such as from any of several tissues of any mammalian source, for example from human tissues.

The terms “mature, full-length KGF,” “long form of KGF,” “FL-KGF,” “native KGF” or “KGF163” refer to the mature polypeptide that contains 163 amino acid residues. The term “KGF fragment” refers to a polypeptide derived from KGF163 that does not include the entire sequence of KGF163. Such a fragment may be a truncated version of the full-length molecule, as well as an internally deleted polypeptide. See, for example, Int'l. Publ. Nos. WO2003016505, WO1995001434, WO1998006844, WO2002062842; U.S. Pat. No. 7,265,089, U.S. Pat. No. 5,773,586, U.S. Pat. No. 5,863,767; or European Pat. Nos. EP0871730B1, or EP1012186B, all of which are herein incorporated by reference in their entireties. The protein sequence of KGF can be found at the Uniprot database under accession number P21781. Two isoforms of human KGF are known, Isoform 1 (Uniprot: P21781-1) and Isoform 2 (Uniprot: P21781-2). The term KGF as used herein encompasses also non-human forms of the protein.

The term “EGF” as used herein refers to full sequence epidermal growth factor and to any fragment of the EGF molecule that retain their biological activity, such as forms truncated at the C-terminal (Calnan et al, Gut 2000, 47 molecules. 622-627) or truncated N-terminal end (Svodoca et al. Biochim Biophys Acta 1994, 1206: 35-41; Shin et al, Peptides 1995, 16: 205-210). The term EGF also encompasses variants with amino acid substitution (Shiah et al, J. Biol Chem 1992, 267: 24034-24040; Lahti et al, FEBS Lett 2011, 585:1135-1139, International Publ. No. WO2007/065 464).

Epidermal growth factor (EGF) is a naturally-occurring, relatively short, single-chain polypeptide, which was first isolated from the mouse submaxillary gland. Both mouse and human epidermal growth factors (the latter one also called urogastrone in some earlier publications) contain 53 amino acids. Thirty-seven of these are identical in the amino acid sequences of mouse epidermal growth factor (mEGF) and human epidermal growth factor (hEGF), as are the relative positions of the three disulfide bonds present in the structure. [Gregory, Nature, 257, 325 (1975); Gregory et al., Hoppe-Seyler's Z. Physiol. Chem., 356, 1765 (1975)]. The polypeptide also exists as a 52 amino acid form (gamma-hEGF) that lacks the C-terminal arginine residue found in OhEGF. The amino acid and nucleotide sequences of hEGF are, for example, disclosed in Hollenberg, “Epidermal Growth Factor-Urogastrone, A Polypeptide Acquiring Hormonal States”; eds., Academic Press, Inc., New York (1979), pp. 69-110; or Urdea et al., Proc. Natl. Acad. Sci. USA. 80, 7461 (1983).

A 48 amino acid containing form of hEGF (lacking C-terminal 5 amino acids) is described in the Japanese Patent Application 86146964, published 8 Feb. 1988 under No. 63003791. The molecule in natural form contains disulfide linkages between residues 6-20, 14-31 and 33-42, and arises from an about 1200 amino acid precursor molecule consisting of eight EGF-like regions [see e.g. Bell et al., Nucleic Acid Research, 14, 21, 8427 (1986)]. A 48 amino acid containing form of rat EGF has been disclosed in the Japanese Patent Application 8736498, published 22 Aug. 1988, under No. 63202387. Both mEGF and hEGF, as well as their known analogs, exhibit similar pharmacological activities, although the extent or spectrum of activity may be different for different materials. In general EGF inhibits the secretion of gastric acid and promotes cell growth; therefore, it is targeted for therapeutic potential as an anti-ulcer agent and in external wound healing. See also, U.S. Pat. No. 6,191,106, U.S. Pat. No. 5,034,375, U.S. Pat. No. 5,102,789, U.S. Pat. No. 5,183,805; U.S. Publ. Nos. US20090081222, US20060014684; or Int'l. Publ. No. WO1992003476, all of which are herein incorporated by reference in their entireties. The term EGF includes also non-human forms of EGF. The sequence of the human EGF protein can be found in the Uniprot database under accession number P01133. Also included in the Uniprot database are the sequences of EGF isoforms such as Isoform 1 (Uniprot: P01133-1), Isoform 2 (Uniprot: P01133-2), Isoform 3 (Uniprot: P01133-3). Natural variants known in the art include variants with substitutions at the following positions: S16R, H151Y, D257Y, L292H, R431K, S638R, M7081, G723R, D784V, M842T, E920V, D981E, L1043F, A1084G

The term “subject” refers to any animal (e.g., a mammal), including, but not limited to humans, non-human primates, rodents, and the like, which is to be the recipient of a particular treatment with a transdermal composition disclosed herein (e.g., a therapeutic composition comprising at least one therapeutic agent in inclusion body form, or a cosmetic composition comprising at least one cosmetic agent in inclusion body form). Typically, the terms “subject” and “patient” are used interchangeably herein in reference to a human subject.

The term “therapeutic agent” as used herein refers to a chemical material or compound that is suitable for topical administration and induces a desired physiological effect. The term “therapeutic agent” encompasses, for example, therapeutic polypeptides (e.g., therapeutic proteins or peptides). By “therapeutic protein” or “therapeutic polypeptide” is meant any naturally or non-naturally occurring protein or polypeptide possessing valuable biological properties that may be useful in the treatment of diseases (e.g., skin diseases) or in preventive medicine by conferring a therapeutic benefit to a host when administered to the host, or when it is expressed in cells of the host. For the purposes of this invention, beneficial or desired clinical results of the therapeutic protein include, but are not limited to, symptom relief, reduction of the extension of the disease, stabilized pathological state (specifically not worsened), delaying or stopping the progression of the disease, improvement or palliation of the pathological state and remission (both partial and total), both detectable and non-detectable.

One of ordinary skill in the art would appreciate that beneficial or desired clinical results are not limited to those enumerated above. In some aspects, the therapeutic agent is an immunogen. Accordingly, in some aspects, the therapeutic compositions disclosed herein comprise vaccines.

As used herein, the term “cosmetic agent” refers to a substance that, for example, a peptide or protein, that aids in the enhancement or protection of the appearance (e.g. color, texture, look, feel, etc.) or odor of a subject's skin. A cosmetic agent may change the underlying structure of the skin. In particular, the term “cosmetic agent” means any substance, as well any component thereof, intended to be rubbed, poured, sprinkled, sprayed, introduced into, or otherwise applied to a subject's body or any part thereof.

Cosmetic agents may include substances that are Generally Recognized as Safe (GRAS) by the US Food and Drug Administration, food additives, and materials used in non-cosmetic consumer products including over-the-counter medications. In some embodiments, cosmetic agents may be incorporated in a cosmetic composition comprising a dermatologically acceptable carrier suitable for topical application to skin.

Cosmetic agents include, for example:
(i) chemicals, compounds, small or large molecules, extracts, formulations, or combinations thereof that are known to induce or cause at least one effect (positive or negative) on skin tissue;
(ii) chemicals, compounds, small molecules, extracts, formulations, or combinations thereof that are known to induce or cause at least one effect (positive or negative) on skin tissue and are discovered, using the provided methods and systems, to induce or cause at least one previously unknown effect (positive or negative) on the skin tissue; and
(iii) chemicals, compounds, small molecules, extracts, formulations, or combinations thereof that are not known have an effect on skin tissue and are discovered, using the provided methods and systems, to induce or cause an effect on skin tissue.

Some examples of cosmetic agents or cosmetically actionable materials can be found, for example, in the PubChem database associated with the National Institutes of Health, USA W (pubchem.ncbi.nlm.nih.gov); the Ingredient Database of the Personal Care Products Council (personalcarecouncil.org); the 2010 International Cosmetic Ingredient Dictionary and Handbook, 13th Edition, published by The Personal Care Products Council; the EU Cosmetic Ingredients and Substances list; the Japan Cosmetic Ingredients List; the Personal Care Products Council, the SkinDeep database (www.cosmeticsdatabase.com); the FDA Approved Excipients List; the FDA OTC List; the Japan Quasi Drug List; the US FDA Everything Added to Food database; EU Food Additive list; Japan Existing Food Additives, Flavor GRAS list; US FDA Select Committee on GRAS Substances; US Household Products Database; the Global New Products Database (GNPD) Personal Care, Health Care, Food/Drink/Pet and Household database (www.gnpd.com); and from suppliers of cosmetic ingredients and botanicals.

The term “therapeutic composition” refers to a preparation comprising a therapeutic agent which is in such form as to permit the biological activity of the active ingredient (e.g., a protein or polypeptide in inclusion body form) to be effective, and which contains no additional components which are unacceptably toxic to a subject to which the composition would be administered. Such composition can be sterile.

The term “cosmetic composition” refers to a preparation comprising a cosmetic agent which is in such form as to permit the desired activity of the active ingredient (e.g., a protein or polypeptide in inclusion body form) to be effective (for example, to aid in the enhancement or protection of the appearance such as color, texture, look, feel, etc., or odor of a subject's skin) and which contains no additional components which are unacceptably toxic to a subject to which the composition would be administered.

An “effective amount” of a therapeutic agent or cosmetic agent in inclusion body form as disclosed herein, is an amount sufficient to carry out a specifically stated purpose. An “effective amount” can be determined empirically and in a routine manner, in relation to the stated purpose. Thus, used herein, the term “effective amount” refers to a dosage sufficient to provide treatment for the condition being treated, or to achieve a certain cosmetic effect (e.g., reduction in wrinkles or increase in skin flexibility). This can vary depending on the subject, the condition and the treatment being effected, or the expected therapeutic and/or cosmetic effect. The exact amount that is required will vary from subject to subject, depending on the subject's species, age, and general condition of the subject, the particular carrier or adjuvant being used, mode of administration, and the like. As such, the effective amount will vary based on the particular circumstances, and an appropriate effective amount can be determined in a particular case by one of ordinary skill in the art using only routine experimentation.

The term “therapeutically effective amount” refers to an amount of a therapeutic agent in inclusion body form as disclosed herein, alone or in combination with another drug, which is effective to “treat” a disease or disorder in a subject or mammal.

The term “cosmetically effective amount” refers to an amount of a cosmetic agent in inclusion body form as disclosed herein, alone or in combination with another drug, which is effective to “improve” a skin condition in a subject or mammal.

The word “label” when used herein refers to a detectable compound or composition which is conjugated or fused directly or indirectly (e.g., via linkers) to a therapeutic agent or cosmetic agent disclosed herein so as to generate a “labeled” therapeutic agent or cosmetic agent. The label can be detectable by itself (e.g., radioisotope labels or fluorescent labels) or, in the case of an enzymatic label, can catalyze chemical alteration of a substrate compound or composition which is detectable.

The term “skin” as used herein includes, for example, the skin on the face, neck, chest, back, arms, hands, legs, and scalp. It is to be understood that administration to mucosal tissue is intended as a possibility as well. The term “skin barrier” as used herein refers to the physical and chemical barrier between the environment and the deeper skin layers posed by the stratum corneum of the skin.

The term “topical administration” as applied to the compositions, methods, and devices of the instant disclosure refers to the application of a therapeutic agent or cosmetic agent in inclusion body form to the skin or to mucosal tissue, for example, for the treatment of various skin conditions or disorders. A “topical composition” is one that is suitable for topical administration.

The term “transdermal” as used herein refers to the delivery of a therapeutic or cosmetic agent in inclusion body form through the skin barrier (e.g. so that at least some portion of the population of therapeutic or cosmetic agent molecules reaches underlying layers of the skin), for example, to reach a location in the skin, under the skin, or at a location distant from the point of application which can be reached, e.g., via the bloodstream. Accordingly, the instant disclosure provides, e.g., “transdermal delivery compositions” (i.e., compositions comprising a therapeutic agent or a cosmetic agent in inclusion body form), “transdermal delivery systems” (i.e., systems comprising a therapeutic agent or a cosmetic agent in inclusion body form), and “transdermal delivery devices/apparatuses” (i.e., devices or apparatuses comprising a therapeutic agent or a cosmetic agent in inclusion body form).

It is not intended that the transdermal delivery be limited to cosmetic agents and therapeutic agents targeted, for example, to the skin or a subcutaneous area of a subject's skin. In this respect, transdermal delivery also encompasses the delivery of therapeutic agent or cosmetic agents to the bloodstream.

### II. Transdermal Delivery of Inclusion Bodies

The instant disclosure provides transdermal delivery methods, compositions, and devices for providing therapeutic agents and cosmetic agents in inclusion body form to a subject in need thereof. Aspects of the invention can be used to transdermally deliver high (or both low and high) molecular weight pharmaceuticals, prophylactics, diagnostics, and cosmetic agents to a subject.

The inventors have demonstrated that large polypeptides such as green-fluorescent protein (GFP), a protein composed of 238 amino acid residues (26.9 kDa), prepared in inclusion body form and applied to a well-established in vitro human skin penetration model are able to penetrate through the stratum corneum and reach deep epidermal layers.

In contrast, most soluble proteins such as soluble GFP are unable to cross the stratum corneum barrier of the skin. These GFP inclusion bodies are spherical or cylindrical entities with average sizes of 300 nm length and 170 nm diameter (Garcia-Fruitos et al. (2009) Advanced Materials 21:4249-4253). It is well known in the art that large proteins in soluble form cannot permeate passively across the skin due to the barrier posed by the stratum corneum, and therefore enhancement techniques are needed to overcome this barrier (Kalluri et al., (2011) AAPS PharmSciTech 12(1):431-441). The finding that nonsolubilized inclusion bodies, highly pure protein deposits in the size range of a few hundred nanometers, can penetrate the skin barrier provides therefore a solution to a long felt need in medicine and cosmetics. U.S. patent application Ser. No. 13/142,295 (published as U.S. Patent Publication No. US 2011-0268773), and U.S. patent application Ser. No. 13/319,772 (published as U.S. Patent Publication No. 2012-0148529), as well as all the references cited in those two U.S. patent applications are herein incorporated by reference in their entireties. In addition, the inventors have shown that proteins such as IL-10 (which can simultaneously function as a therapeutic agent by reducing inflammation, and as a cosmetic agent by reducing redness and swelling associated with inflammation) in inclusion body form can penetrate the skin barrier and be effective in human psoriatic skin samples.

The instant disclosure provides methods for delivering a cosmetic agent across the skin barrier comprising applying to the skin of a subject a cosmetic composition comprising at least one cosmetic agent in inclusion body form, wherein the at least one cosmetic agent crosses the skin barrier in inclusion body form. Thus, after the intact IB is applied to the skin of the subject, it permeates through the skin, and even when located deep in the skin the IB still maintains its structural integrity. These methods can also be applied to the delivery of therapeutic agents in inclusion body form across the skin barrier. Accordingly, the instant disclosure also provides methods for delivering a therapeutic agent across the skin barrier comprising applying a therapeutic composition to the skin of a subject in need thereof, wherein the therapeutic comprises at least one therapeutic agent in inclusion body form, and wherein the at least one therapeutic agent crosses the skin barrier in inclusion body form.

The therapeutic and cosmetic agents delivered in inclusion body form can be used, for example, to treat skin conditions. For example, cosmetic agents can be delivered to improve skin conditions such as wrinkles, sun damage, or cellulite. In some aspects, cosmetic agents can be delivered to prevent skin conditions such as sun damage (e.g., when the cosmetic agent is applied as part of a sunscreen). In this respect, the instant disclosure provides a method for treating a skin condition in a subject in need thereof comprising topically applying a cosmetically effective amount of a cosmetic composition comprising at least one cosmetic agent in inclusion body form, and a dermatologically acceptable carrier to the skin of the subject so as to improve the skin condition of the subject. In some cases, therapeutic agents can be delivered to improve skin conditions such as inflammation (e.g., caused by infection or immune reactions, including autoimmune reactions) or cancer. Accordingly, the instant disclosure provides methods for treating a skin condition in a subject in need thereof comprising topically applying a therapeutically effective amount of a therapeutic composition comprising at least one therapeutic agent in inclusion body form, and a pharmaceutically acceptable carrier to the skin of the subject so as to improve the skin condition of the subject.

Since high molecular size molecules such as proteins are generally unable to permeate through the skin barrier, providing therapeutic and cosmetics agents (e.g., proteins) in inclusion body form as disclosed herein can be used to enhance the penetrative capability of generally non-skin permeant therapeutic and cosmetics agents (e.g., proteins). Accordingly, the instant disclosure provides a method of enhancing penetration of the skin by a cosmetic agent comprising applying to the skin of a subject a cosmetic composition comprising at least one cosmetic agent in inclusion body form, wherein the penetration of the cosmetic agent is increased with respect to the penetration of the same cosmetic agent in soluble form. This improvement in penetration can also be applied to therapeutic agents, e.g., therapeutic proteins. Therefore, the instant disclosure also provides a method of enhancing penetration of the skin by a therapeutic agent comprising applying to the skin of a subject a therapeutic composition comprising at least one therapeutic agent in inclusion body form, wherein the penetration of the therapeutic agent is increased with respect to the penetration of the same therapeutic agent in soluble form.

The capability of inclusion bodies to stimulate tissue regeneration and to stimulate eukaryotic cell proliferation in vitro has been disclosed in U.S. patent application Ser. No. 13/142,295 (published as U.S. Patent Publication No. US 2011-0268773) which is herein incorporated by reference in its entirety. Accordingly, the instant disclosure provides a method of stimulating tissue regeneration, comprising applying to the skin of a subject at least one cosmetic agent or therapeutic agent in inclusion body form, wherein the inclusion body penetrates the skin barrier and reaches said tissue and stimulates its regeneration. Also provided is a method of stimulating eukaryotic cell proliferation, comprising applying to the skin of a subject at least at least one cosmetic agent or therapeutic agent in isolated inclusion body form, wherein the inclusion body penetrates the skin barrier and stimulates eukaryotic cell proliferation.

The inclusion bodies disclosed herein can be incorporated into a transdermal delivery system (for example, a patch, a spray, a swab, a sponge, a stick, a shampoo, etc.). Hence, the instant disclosure also provides a method of making a transdermal delivery system comprising (i) providing at least one cosmetic agent or a therapeutic agent in inclusion body form, and (ii) mixing the inclusion body with a carrier, thereby making the transdermal delivery system.

In some aspects, the inclusion body is insoluble. In other aspects, the inclusion body is soluble, but it has not been solubilized. In yet other aspects, the inclusion body is partially solubilized. In some cases, inclusion bodies can be partially solubilized, e.g., by one or more washes with solutions containing solubilizing agents such as detergents or organic solvents. Partial solubilization can be used, for example, to remove lipid membranes surrounding the inclusion body, or to strip host cell contaminants adhered to the outer layers of the inclusion bodies. In some aspects, partial solubilization of the outer layers can be used to increase the purity of the inclusion bodies.

In some aspects, the inclusion body is in particulate form. In particular, the inclusion bodies disclosed herein can have a particle size between 20 nm and 1500 nm. The particle size refers to the diameter of the particles where they are substantially spherical. The particles may be non-spherical, in which case the particle size range can refer to the equivalent diameter of the particles relative to spherical particles.

In some aspects, the average inclusion body particle size is about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1050 nm, about 1100 nm, about 1150 nm, about 1200 nm, about 1250 nm, about 1300 nm, about 1350 nm, about 1400 nm, about 1450 nm, or about 1500 nm.

In some aspects, the average inclusion body particle size is between about 100 nm and about 200 nm. In some aspects, the average inclusion body particle size is between about 200 nm and about 300 nm. In some aspects, the average inclusion body particle size is between about 300 nm and about 400 nm. In some aspects, the average inclusion body particle size is between about 400 nm and about 500 nm. In some aspects, the average inclusion body particle size is between about 500 nm and about 600 nm. In some aspects, the average inclusion body particle size is between about 600 nm and about 700 nm. In some aspects, the average inclusion body particle size is between about 700 nm and about 800 nm. In some aspects, the average inclusion body particle size is between about 800 nm and about 900 rm. In some aspects, the average inclusion body particle size is between about 900 nm and about 1000 nm. In some aspects, the average inclusion body particle size is between about 1000 nm and about 1100 nm. In some aspects, the average inclusion body particle size is between about 1100 nm and about 1200 nm. In some aspects, the average inclusion body particle size is between about 1200 nm and about 1300 nm. In some aspects, the average inclusion body particle size is between about 1300 nm and about 1400 nm. In some aspects, the average inclusion body particle size is between about 1400 nm and about 1500 nm.

In some aspects, the average inclusion body particle size has a particle size between about 150 nm and about 300 nm. In some aspects, the average inclusion body particle size has a particle size between about 100 nm and about 500 rm.

In some aspects, the inclusion body is in hydrated amorphous form. In some aspects, after penetrating the skin barrier, the inclusion body can be internalized by a target cell. In some aspects, the target cell is an epidermal cell. In other aspects, the target cell is a non-epidermal cell. In some aspects, the target cell is, for example, a neuron, a muscle cell, an adipocyte, a melanocyte, a hair follicle cell, a sweat gland cell, a sebaceous gland cell, a cell in a blood vessel, a keratinocyte, a Merkel cell, a Langerhans cell, or a combination thereof. The list provided is not limiting.

In some aspects, the inclusion body can penetrate at least one skin layer, generally the cornified layer (stratum corneum), although in other aspects inclusion bodies can penetrate deeper in the skin. Accordingly, in some aspects the inclusion body can penetrate the translucent layer (stratum lucidum), the granular layer (stratum granulosum), the spinous layer (stratum spinosum) o basal/germinal layer (stratum basale/germinativum). In some aspects, the inclusion can penetrate deeper than the epidermis. In some aspects, the therapeutic agent in the inclusion body can penetrate the skin and be delivered to the bloodstream.

In some aspects, the inclusion body can penetrate an epithelial tissue layer, for exempla a layer of one of the epithelial tissues described in TABLE 1.

TABLE 1
Types of epithelial tissue

| | | Epithelial Tissue | |
|---|---|---|---|
| System | Tissue | Type | Subtype |
| circulatory | blood vessels | Simple squamous | endothelium |
| digestive | ducts of | Stratified columnar | — |
| | submandibular | | |
| | glands | | |
| digestive | attached gingiva | Stratified squamous, | — |
| | | keratinized | |
| digestive | dorsum of tongue | Stratified squamous, | — |
| | | keratinized | |
| digestive | hard palate | Stratified squamous, | — |
| | | keratinized | |
| digestive | esophagus | Stratified squamous, | — |
| | | non-keratinized | |
| digestive | stomach | Simple columnar, | gastric |
| | | non-ciliated | epithelium |
| digestive | small intestine | Simple columnar, | intestinal |
| | | non-ciliated | epithelium |
| digestive | large intestine | Simple columnar, | intestinal |
| | | non-ciliated | epithelium |
| digestive | rectum | Simple columnar, | — |
| | | non-ciliated | |
| digestive | anus | Stratified squamous, | — |
| | | non-keratinized | |
| | | superior to | |
| | | Hilton's white | |
| | | line | |
| | | Stratified | |
| | | squamous, kera- | |
| | | tinized inferior to | |
| | | Hilton's white line | |
| digestive | gallbladder | Simple columnar, | — |
| | | non-ciliated | |
| endocrine | thyroid follicles | Simple cuboidal | — |
| nervous | ependymal | Simple cuboidal | — |
| lymphatic | lymph vessel | Simple squamous | endothelium |
| integumentary | skin - dead | Stratified squamous, | — |
| | superficial layer | keratinized | |
| integumentary | sweat gland ducts | Stratified cuboidal | — |
| integumentary | mesothelium of | Simple squamous | mesothelium |
| | body cavities | | |
| reproductive - | ovaries | Simple cuboidal | germinal |
| female | | | epithelium |
| | | | (female) |
| reproductive - | Fallopian tubes | Simple columnar, | — |
| female | | ciliated | |
| reproductive - | endometrium | Simple columnar, | — |
| female | (uterus) | ciliated | |
| reproductive - | cervix | Simple columnar | — |
| female | (endocervix) | | |
| reproductive - | cervix | Stratified squamous, | — |
| female | (ectocervix) | non-keratinized | |
| reproductive - | vagina | Stratified squamous, | — |
| female | | non-keratinized | |
| reproductive - | labia majora | Stratified squamous, | — |
| female | | keratinized | |
| reproductive - | tubuli recti | Simple cuboidal | germinal |
| male | | | epithelium |
| | | | (male) |
| reproductive - | rete testis | Simple cuboidal | — |
| male | | | |
| reproductive - | ductuli | Pseudostratified | — |
| male | efferentes | columnar | |
| reproductive - | epididymis | Pseudostratified | — |
| male | | columnar, with | |
| | | stereocilia | |
| reproductive - | vas deferens | Pseudostratified | — |
| male | | columnar | |
| reproductive - | ejaculatory duct | Simple columnar | — |
| male | | | |
| reproductive - | bulbourethral | Simple columnar | — |
| male (gland) | glands | | |
| reproductive - | seminal vesicle | Pseudostratified | — |
| male (gland) | | columnar | |
| respiratory | oropharynx | Stratified squamous, | — |
| | | non-keratinized | |
| respiratory | larynx | Pseudostratified | respiratory |
| | | columnar, ciliated | epithelium |
| respiratory | larynx - True | Stratified squamous, | — |
| | vocal cords | non-keratinized | |
| respiratory | trachea | Pseudostratified | respiratory |
| | | columnar, ciliated | epithelium |
| respiratory | respiratory | Simple cuboidal | — |
| | bronchioles | | |
| sensory | cornea | Stratified squamous, | corneal |
| | | non-keratinized | epithelium |
| sensory | nose | Pseudostratified | olfactory |
| | | columnar | epithelium |
| urinary | kidney - proximal | Simple cuboidal, | — |
| | convoluted tubule | with microvilli | |
| urinary | kidney - | Simple squamous | — |
| | ascending | | |
| | thin limb | | |
| urinary | kidney - distal | Simple cuboidal, | — |
| | convoluted tubule | without microvilli | |
| urinary | kidney - | Simple cuboidal | — |
| | collecting duet | | |
| urinary | renal pelvis | Transitional | urothelium |
| urinary | ureter | Transitional | urothelium |
| urinary | urinary bladder | Transitional | urothelium |
| urinary | prostatic urethra | Transitional | urothelium |
| urinary | membranous | Pseudostratified | — |
| | urethra | columnar, non-ciliated | |
| urinary | penile urethra | Pseudostratified | — |
| | | columnar, non-ciliated | |
| urinary | external urethral | Stratified squamous | — |
| | orifice | | |

In some aspects, the cosmetic agent or therapeutic agent in inclusion body form comprises a polypeptide. In some aspects, the polypeptide is biologically active; however, in other aspects, the polypeptide is a prodrug. As used herein the term “prodrug” means a therapeutic or cosmetic agent as disclosed herein which is a labile derivative compound of a parent agent which when administered to a subject in vivo becomes cleaved by chemical and/or enzymatic hydrolysis thereby forming the parent therapeutic or cosmetic agent such that a sufficient amount of the agent intended to be delivered to the subject is available for its intended therapeutic or cosmetic use in a sustained release manner.

The term “labile” as used herein refers to the capacity of the prodrug to undergo enzymatic and/or chemical cleavage in vivo thereby forming the parent agent. The term “sustained release” (referred sometimes in the art as “sustained delivery” or “extended release”) indicates that the prodrug provides release of the parent therapeutic or cosmetic agent by any mechanism including slow first-order kinetics of absorption or zero-order kinetics of absorption, such that the parent therapeutic or cosmetic agent which is released from the prodrug provides a longer duration of action than the duration of action of the parent therapeutic or cosmetic agent when administered alone (i.e. not as a prodrug).

In some aspects, the polypeptide is a recombinant polypeptide or a fragment thereof, a natural polypeptide or a fragment thereof, or a chemically synthesized polypeptide. Methods for recombinant production of proteins to generate inclusion bodies are known in the art. Specific methods for production, purification, and characterization of inclusion bodies are disclosed in detail below. Methods for chemical synthesis of peptides are also well known in the art. Accordingly, the term inclusion body as used herein also includes insoluble protein precipitates or nanoparticles produced, e.g., from chemically synthesized peptides and proteins, from peptides and proteins obtained from natural sources, or from artificial virus-like particles (see, e.g., Domingo-Espin et al. (2011) Nanomedicine 6:1047-1061; Domingo-Espin et al. (2010) J. Biotechnol. 150:437-438).

In some aspects, the polypeptide is a fusion protein or a protein conjugate. For example, the polypeptide can comprise a therapeutic or cosmetic agent chemically conjugated to another protein, for example, an inclusion body-inducing peptide. Conjugation can be conducted using derivatizable groups and methods known in the art. Selectively derivatizable groups are well known in the art, such as an amino group, sulfhydryl group, pendant oxyamino, or other nucleophilic groups. Derivatizable groups can be joined to a polypeptide chain via one or more linkers. Ligands (e.g., additional therapeutic agents or cosmetic agents, detectable labels, half-life extending polymers, etc.) can be attached to the derivatizable groups using the appropriate attachment chemistry. This coupling chemistry can include, for example, amide, urea, thiourea, oxime, aminoacetylamide, etc. Suitable crosslinkers for conjugation include those that are heterobifunctional, having two distinctly reactive groups separated by an appropriate spacer (e.g., m-maleimidobenzoyl-N-hydroxysuccinimidc ester) or homobifunctional (e.g., disuccinimidyl suberate). Such crosslinkers are available, for example, from Pierce Chemical Company, Rockford, II. Additional bifunctional coupling agents include N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some aspects, the polypeptide is chimeric, i.e., the polypeptide is the result of fusing or chemically conjugating at least two proteins or fragments thereof. In some aspects, such proteins are obtained from the same species, although in other cases the components of the chimeric protein can be obtained from proteins from different species. In some aspects, the recombinant polypeptide is expressed in a cell, for example, bacteria, yeasts, insect cells, and mammalian cells. A person skilled in the art would understand that any cell expression system, cell-free expression system, or alternative method to obtain inclusion bodies can be applied to obtain inclusion bodies to use according to the methods disclosed herein.

In some aspects, the polypeptide is conjugated to a protein purification tag or a detectable label, for example, a visualization tag. In some aspects, the protein purification tag is a His6-tag. In some aspects, the visualization tag is a fluorescent tag. Useful detectable labels include fluorescent compounds (e.g., fluorescein, fluorescein isothiocyanate, rhodamine, 5-dimethylamine-1-napthalenesulfonyl chloride, phycoerythrin, lanthanide phosphors and the like), enzymes that are useful for detection (e.g., horseradish peroxidase, β-galactosidase, luciferase, alkaline phosphatase, glucose oxidase and the like), radioactive labels, or epitopes recognized by a secondary reporter (e.g., leucine zipper pair sequences, binding sites for secondary antibodies, metal binding domains, epitope tags, etc.). In some aspects, detectable labels can be attached by spacer arms of various lengths to reduce potential steric hindrance.

In some specific aspects, the cosmetic agent is a protein selected from the group consisting of IL-10, EGF, KGF, VEGF, or a combination thereof. In other aspects, the therapeutic agent is a protein selected from the group consisting of IL-10, EGF, KGF, VEGF, or a combination thereof.

### III. Cosmetic Agents and Therapeutic Agents

The methods, compositions, and devices disclosed herein can use therapeutic agents and/or cosmetic agents in inclusion body form, wherein said agents are, for example, low or high (or both low and high) molecular weight pharmaceuticals, prophylactics, diagnostics, and cosmetic agents. The therapeutic agents and cosmetic agents disclosed herein include, for example, nucleic acids, polypeptide, peptides, modified peptides, small molecules, immunogenic preparations, and the like.

The inclusion bodies disclosed herein can be used, for example, to administer hormones, anesthetics, collagen preparations, cardiovascular pharmaceutical compounds, anti-infective compounds (e.g., antibiotics and antiviral compounds), diabetes-related treatments, immunogenic compositions, vaccines, immune response modifiers, enzyme inhibitors, analgesics, migraine therapies, sedatives, imaging and contrast compounds. These examples are provided to disclose that aspects of the invention can be used to transdermally deliver both low and high molecular weight compounds and it should be understood that many other molecules can be effectively delivered to the body, using the molecules described herein, in amounts that are therapeutically, prophylactically, or cosmetically beneficial.

In some aspects, the inclusion bodies comprise polypeptides such as erythropoietin (EPO), corticotropin-releasing hormone (CRH), growth hormone-releasing hormone (GHRH), gonadotropin-releasing hormone (GnRH), thyrotropin-releasing hormone (TRH), prolactin-releasing hormone (PRH), melanotropin-releasing hormone (MRH), prolactin-inhibiting hormone (PIH), somatostatin, adrenocorticotropic hormone (ACTH), somatotropin or growth hormone (GH), luteinizing hormone (LH), follicle-stimulating hormone (FSH), thyrotropin (TSH or thyroid-stimulating hormone), prolactin, oxytocin, antidiuretic hormone (ADH or vasopressin), melatonin, Müllerian inhibiting factor, calcitonin, parathyroid hormone, gastrin, cholecystokinin (CCK), secretin, insulin-like growth factor type I (IGF-I), insulin-like growth factor type II (IGF-II), atrial natriuretic peptide (ANP), human chorionic gonadotropin (hCG), insulin, glucagon, somatostatin, pancreatic polypeptide (PP), leptin, neuropeptide Y, renin, angiotensin I, angiotensin II, factor VIII, factor IX, tissue factor, factor VII, factor X, thrombin, factor V, factor XI, factor XIII, interleukin 1 (IL-1), Tumor Necrosis Factor Alpha (TNF-α), interleukin 6 (IL-6), interleukin 8 (IL-8), interleukin-10 (IL-10), interleukin 12 (IL-12), interleukin 16 (IL-16), interferons alpha, beta, gamma, nerve growth factor (NGF), platelet-derived growth factor (PDGF), transforming growth factor beta (TGF-beta), bone morphogenetic proteins (BMPs), fibroblast growth factor-(FGF), epidermal growth factor (EGF), vascular endothelial growth factor (VEGF), granulocyte colony-stimulating factor (G-CSF), glial growth factor, keratinocyte growth factor (KGF), endothelial growth factor, alpha-1 antitrypsin, granulocyte-macrophage colony-stimulating factor (GM-CSF), cyclosporine, fibrinogen, lactoferrin, tissue-type plasminogen activator (tPA), chymotrypsin, immunoglobins, hirudin, superoxide dismutase, imiglucerase, dihydrofolate reductase (DHFR), catalase, or chaperones (e.g., Hsp70).

In specific aspects, the proteins in the inclusion bodies comprise EGF, KGF, or VEGF, or fragments, variants, or derivatives thereof which retain at least part of the therapeutic, prophylactic, or cosmetic properties of the native protein.

In one aspect, the inclusion body comprises a single therapeutic, prophylactic, or cosmetic protein selected from the group consisting of EGF, a EGF fragment, an EGF variant, an EGF derivative, and a combination thereof.

In another aspect, the inclusion body comprises a single therapeutic, prophylactic, or cosmetic protein selected from the group consisting of KGF, a KGF fragment, a KGF variant, a KGF derivative, or a combination thereof.

In yet another aspect, the inclusion body comprises a single therapeutic, prophylactic, or cosmetic agent consisting of VEGF, a VEGF fragment, a VEGF variant, a VEGF derivative, or a combination thereof.

Numerous growth factors can be used in topical skin formulations (e.g., creams). Topical skin formulations (e.g., creams) can containing a single growth factor or multiple growth factors and cytokines. Such formulations can also contain soluble collagen, matrix proteins and antioxidants to neutralise free radicals. Examples of growth factors that can be included in topical skin formulations are listed in TABLE 2 (below).

TABLE 2
Growth factors and their function in topical skin formulations.

| Growth factor | Function |
|---|---|
| Transforming growth factor | Stimulate collagen secretion |
| beta (TGF-B) | |
| Vascular endothelial growth | Stimulate new blood vessel formation |
| factor (VEGF) | |
| Hepatocyte growth factor | Stimulate new blood vessel formation |
| (HGF) | |
| Keratinocyte growth factor | Stimulate epithelial cell growth |
| (KGF) | |
| Interleukins (IL-6, IL-7, | Reduce inflammation |
| IL-8, IL-10) | |
| Basic fibroblast growth | Promote formation of new blood vessels |
| factor (bFGF) | |
| Insulin-like growth factor 1 | Promote cell growth and multiplication |
| (IGF1) | |
| Platelet-derived growth factor | Regulate cell growth and division |
| AA (PDGF-AA) | |
| Transforming growth factors | Stimulate collagen secretion |
| (TGF-B2 & B3) | |
| Granulocyte monocyte colony | Increase number of white blood cells |
| stimulating factor | |

When incorporated in a topical formulation, the growth factors can reverse, prevent or treat the signs and symptoms of (i) intrinsic ageing mediated by the process of natural ageing, and/or (ii) extrinsic ageing mediated by environmental factors. Effects of intrinsic ageing that can be reversed, prevented, or treated include the tendency for cells to stop proliferation or division, decrease of amount of collagen in the skin, degradation of collagen in the skin, dermal thinning, loss in elasticity and increase in skin laxity, etc. Effects of extrinsic ageing (e.g., caused by environmental factors such as air dryness, or UV radiation, or artificial factors such as chemical peelings or laser therapy) that can be reversed, prevented, or treated include coarse wrinkling, broken blood vessels, skin laxity, dryness, prominence of pores, skin discoloration, uneven skin tone, etc. Accordingly, in some aspects, topical formulations comprising growth factors (e.g., VEGF, KGF, or EGF) can be used to reduce the appearance of file lines and/or wrinkles, improve the appearance of age spots, even out pigmentation, reduce skin roughness, improve skin texture, improve skin elasticity, improve skin smoothness, increase skin tightness, or combinations thereof. See, e.g., Mehta et al. (2007) Dermatologic Therapy 20:350-359; Sundaram et al. (2009) J. Drugs Dermatol. 8:4-13; Michael et al. (2007) JH. Drugs Dermatol. 6:197-202, which are herein incorporated by reference in their entireties.

As used herein, the term “growth factor” also includes growth factor mimicking peptides. Growth factor ingredients registered on CTFA (Cosmetic Toiletry, Fragrance Association) include, for example, EFG (anti-aging), IF-1 (anti-aging, hair-care), bFGF (anti-aging, hair-care), TRX (anti-aging, anti-pigmentation, hair-care), KGF (anti-aging, hair-care), SCF (hair-care), TGF-beta3 (hair-care), IL10 (anti-inflammation), PDGF (anti-aging), VEGF (hair-care), FGFIO (hair-care), aFGF (anti-aging, hair-care), TGF-alpha (anti-aging), IL-4 (anti-inflammation), Thymosin-beta4 (anti-aging, hair-care), Noggin (hair-care), hNGF (hair-care), etc. Growth factor mimicking peptides registered on CTFA include, for example, CG-IDP2™ (anti-aging, hair-care), CG-IDP3™ (anti-aging), CG-IDP4FM (anti-aging), CG-IDP5™ (anti-aging, hair-care), CG-EDPI™ (anti-aging), ALOPECTIN™ (hair-care), RETARDRIN™ (hair-care), REJULINE™ (anti-aging, hair-care), etc.

Many different therapeutic agents or cosmetic agents in inclusion body form can be incorporated into the various transdermal delivery compositions, systems, and devices described herein (and used according to the methods disclosed herein). As used herein, the term “transdermal delivery compositions” encompasses both the therapeutic compositions and the cosmetic compositions of the instant disclosure. Low molecular weight and high molecular weight therapeutic or cosmetic agents can be effectively delivered transdermally using an aspect of the instant disclosure.

A transdermal delivery composition comprising a therapeutic or cosmetic agent in inclusion body form described herein can provide a therapeutically, prophylactically, diagnostically, or cosmetically beneficial amount of a therapeutic or cosmetic agent having a molecular weight of 50 daltons to 2,000,000 daltons or less. That is, a transdermal delivery composition described herein, preferably, provides a delivered a therapeutic or cosmetic agent having a molecular weight of less than or equal to or greater than 50, 100, 200, 500, 1,000, 1,500, 2,000, 2,500, 3,000, 3,500, 4,000, 4,500, 5,000, 5,500, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, 45,000, 46,000, 47,000, 48,000, 49,000, 50,000, 51,000, 52,000, 53,000, 54,000, 55,000, 56,000, 57,000, 58,000, 59,000, 60,000, 61,000, 62,000, 63,000, 64,000, 65,000, 66,000, 67,000, 68,000, 69,000, 70,000, 75,000, 80,000, 85,000, 90,000, 95,000, 100,000, 125,000, 150,000, 175,000, 200,000, 225,000, 250.000, 275,000, 300,000, 350,000, 400,000, 450,000, 500,000, 600,000, 700,000, 800,000, 900,000, 1,000,000, 1,500,000, 1,750,000, and 2,000,000 daltons.

In some aspects, amino acids, peptides, nucleotides, nucleosides, and nucleic acids are transdermally delivered in inclusion body form to cells in the body using an aspect of the transdermal delivery compositions and methods described herein. That is, any peptide or polypeptide having at least, less than, more than, or equal to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 200, 300, 400, 500, 600, 700, 800, 900, 1000, 1500, 2000, 2500, 3000, 3500, 4000, 4500, 5000, 7000, or 10,000 amino acids can be incorporated into a transdermal delivery composition, system, or device described herein (and used according to the methods disclosed herein) and said delivered therapeutic or cosmetic agent can be delivered to cells in the body shortly after application of the composition. These peptide or polypeptides can be used, for example, to stimulate an immune response, reduce inflammation, promote wound healing, induce collagen synthesis, etc.

The peptides or polypeptides disclosed herein also include peptide hormones. Non-limiting examples of peptide hormones that are delivered agents in certain aspects include oxytocin, vasopressin, melanocyte-stimulating hormone, corticotropin, lipotropin, thyrotropin, growth hormone, prolactin, luteinizing hormone, human chorionic gonadotropin, follicle stimulating hormone, corticotropin-releasing factor, gonadotropin-releasing factor, prolactin-releasing factor, prolactin-inhibiting factor, growth-hormone releasing factor, somatostatin, thyrotropin-releasing factor, calcitonin, calcitonin gene-related peptide, parathyroid hormone, glucagon-like peptide 1, glucose-dependent insulinotropic polypeptide, gastrin, secretin, cholecystokinin, motilin, vasoactive intestinal peptide, substance P, pancreatic polypeptide, peptide tyrosine tyrosine, neuropeptide tyrosine, amphiregulin, insulin, glucagon, placental lactogen, relaxin, inhibin A, Inhibin B, Endorphins, angiotensin II, or atrial natriuretic peptide.

Any nucleotide or nucleoside, modified nucleotide or nucleoside, or nucleic acid having at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 75, 100, 125, 150, 200, 300, 400, 500, 600, 700, 800, 900, 1000, 1500, 2000, 2500, 3000, 3500, 4000, 4500, 5000, 7000, or 10,000 or more nucleotides can be incorporated into a transdermal delivery composition, system, or device described herein (and used according to the methods disclosed herein) and said delivered therapeutic agent or cosmetic agent can be delivered to cells in the body shortly after application of the composition. These nucleotides or nucleosides can also be used, for example, to stimulate an immune response, reduce inflammation, promote wound healing, or induce collagen synthesis.

Several nucleic acid immunogens and/or vaccines and therapies are known in the art and can be useful as delivered agents in aspects of the transdermal delivery compositions, methods, systems, and devices disclosed herein. Several nucleic acid immunogens that induce an immune response (both humoral and cellular) upon administration to a host have been described. DNA vaccines for several viruses, as well as for tumors, are known. Those skilled in the art will appreciate that nucleic acid immunogens contain essential regulatory elements such that upon administration to a host, the immunogen is able to direct host cellular machinery to produce translation products encoded by the respective delivered nucleic acids. As used herein, an immunogen is considered a therapeutic agent, and a composition comprising an immunogen is considered a therapeutic composition.

In addition to low molecular weight delivered agents and medium molecular weight delivered agents, several high molecular weight delivered agents (e.g., glycoproteins) can be delivered to cells in the body by using an aspect of the transdermal delivery compositions, methods, systems, and devices disclosed herein. Glycoproteins are high molecular weight compounds, which are generally characterized as conjugated proteins containing one or more heterosaccharides as prosthetic groups. Several forms of glycoproteins are found in the body. For example, many membrane bound proteins are glycoproteins, the substances that fill the intercellular spaces (e.g., extracellular matrix proteins) are glycoproteins, and the compounds that compose collagens, proteoglycans, mucopolysaccharides, glycosaminoglycans, and ground substance are glycoproteins. A transdermal delivery system that can administer therapeutic or cosmetic agents in inclusion body form comprising glycoproteins to cells of the body has several therapeutic and cosmetic uses, including but not limited to, the restoration of skin elasticity and firmness (e.g., reduction in the appearance of fine lines and wrinkles) and the restoration of flexible and strong joints (e.g., caused by increase water retention in joints by transdermal delivery of proteoglycans).

Cosmetic peptides known in the art can be delivered in the transdermal delivery compositions, methods, system, and devices disclosed for herein. For example, cosmetic peptides are disclosed in U.S. Pat. Publ. Nos. US2009/0136595; US2010/0196302; US2005/0226839; US2009/0155317; US2006/0293227; US2009/0143295; US2007/0110686; US2011/0305735, US2010/0098769, US2009/0155317, US2011/0195102, US 2012/0021029, US 2012/0121675, and U.S. Pat. No. 7,943,156, U.S. Pat. No. 7,022,668, U.S. Pat. No. 8,114,439, U.S. Pat. No. 7,473,679, U.S. Pat. No. 6,875,744, U.S. Pat. No. 6,333,042, and U.S. Pat. No. 7,015,192.

Therapeutic peptides that can be delivered according to the instant disclosure comprise, for example, insulins, exendins and derivatives, e.g., lixisenatide (e.g., those disclosed in U.S. Pat. No. 6,989,366, U.S. Pat. No. 7,297,761, U.S. Pat. No. 7,115,569, U.S. Pat. No. 7,138,375, and U.S. Pat. No. 6,956,026, and U.S. Publ. No. US 2005/0215469), muscle relaxant peptides (e.g., those disclosed in US2009/0226387), anti-tumor peptides (e.g., those disclosed in US2003/0109437, US2008/0027005, U.S. Pat. No. 7,241,738); anti-bacterial peptides (e.g., those disclosed in US2002/0035061, US2003/0232750, U.S. Pat. No. 6,503,881, U.S. Pat. No. 5,994,306, U.S. Pat. No. 7,001,983); neuroexocytosis inhibiting peptides (e.g., those disclosed in US2010/0021510, US2008/0241881, US2011/0305735, etc. In some aspects, therapeutic peptides and proteins can be delivered for cosmetic purposes, for example, botulinum toxins, variants, and fragments thereof (e.g., botulinum neurotoxin A or the molecules disclosed in U.S. Pat. No. 5,837,265) or peptides mimicking the action of botulinum neurotoxins (e.g., those disclosed in US2010/0021510, or U.S. Pat. No. 7,473,679). Different cosmetic products aimed at the inhibition of the neuromuscular junction at a synaptic level to avoid the appearance or to soften expression lines can be administered using the compositions and methods disclosed herein. For example, European patents EP 1180524 B1 and PCT Publication No. WO09734620 describe the use of peptides derived from the protein SNAP-25 which act presynaptically competing with SNAP-25 in the formation of the SNARE complex, causing a reduction in the release of ACh and inhibiting the neuronal transmission in the neuromuscular junction.

European Publication No. EP 1809652 A2 describes antagonist peptides of AChRs which act post-synaptically with a mechanism of action similar to waglerin-1 to block the nerve transmission and prevent the appearance of wrinkles. The active cosmetic pentapeptide-3 also acts post-synaptically by inhibiting AChRs, with a mechanism of action similar to tubocurarine to block the nerve transmission and prevent the appearance of wrinkles.

### IV. Therapeutic and Cosmetic Compositions

The therapeutic and cosmetic compositions disclosed herein can comprise at least one therapeutic agent and/or at least one cosmetic agent, respectively, and further comprise a carrier (generally, a dermatologically acceptable carrier). Accordingly, the present disclosure provides a topical cosmetic composition comprising at least one cosmetic agent in isolated inclusion body form, wherein said inclusion body can penetrate the skin barrier, and wherein, in some aspects, such cosmetic composition comprises at least one carrier.

Also provided is a topical therapeutic composition comprising at least one therapeutic agent in isolated inclusion body form, wherein said inclusion body can penetrate the skin barrier, and wherein, in some aspects, such therapeutic composition comprises at least one carrier.

Thus, in addition to a therapeutic or cosmetic agent in inclusion body form disclosed above, the therapeutic or cosmetic compositions described herein can further comprise, for example, carriers and adjuvants such as water (distilled, deionized, filtered, or otherwise prepared), alcohols, nonionic solubilizers, or emulsifiers. Suitable hydrophilic components include, but are not limited to, water, ethylene glycol, propylene glycol, dimethyl sulfoxide (DMSO), dimethyl polysiloxane (DMPX), oleic acid, caprylic acid, isopropyl alcohol, 1-octanol, ethanol (denatured or anhydrous), and other pharmaceutical grade or absolute alcohols.

Carriers such as alcohol, water, and other aqueous adjuvants are not present in some formulations of the transdermal delivery compositions described herein. Other materials can also be components of a transdermal delivery composition of the invention including fragrance, creams, ointments, colorings, and other compounds so long as the added component does not deleteriously affect transdermal delivery of the delivered therapeutic or cosmetic agent in inclusion body form.

As used herein, the term “carrier” refers to molecules (e.g., diluents, adjuvants, excipients or vehicles) with which an agent (e.g., a therapeutic and/or cosmetic agent) is administered to a subject, enhancing the in vivo and/or in vitro stability of the agent, to prevent a decrease in the physiological activity of an agent, or combinations thereof.

In the context of the present disclosure, the term “dermatologically acceptable carriers” refers to carriers suitable for use in the therapeutic and/or cosmetic compositions disclosed herein should be safe for use in contact with human skin tissue. Suitable carriers can include water and/or water miscible solvents. The therapeutic and cosmetic compositions for transdermal delivery of therapeutic or cosmetic agents in inclusion body form disclosed herein can comprise from about 1% to about 95% by weight of water and/or water miscible solvent. The composition may comprise from about 1%, 3%, 5%, 10%, 15%, 20%, 25%, 30%, 35%, 40%, 45%, 50%, 55%, 60%, 65%, 70%, 75%, 80%, or 85% to about 90%, 85%, 80%, 75%, 70%, 65%, 60%, 55%, 50%, 45%, 40%, 35%, 30%, 25%, 20%, 15%, 10%, or 5% water and/or water miscible solvents. Suitable water miscible solvents include monohydric alcohols, dihydric alcohols, polyhydric alcohols, glycerol, glycols, polyalkylene glycols such as polyethylene glycol, and mixtures thereof. When the transdermal delivery compositions disclosed herein are in emulsion form, water and/or water miscible solvents are carriers typically associated with the aqueous phase.

Suitable carriers also include oils. The transdermal delivery compositions disclosed herein can comprise from about 1% to about 95% by weight of one or more oils. The compositions may comprise from about 0.1%, 0.5%, 1%, 2%, 5%, 10%, 15%, 20%, 25%, 30%, 35%, 40%, 45%, 50%, 55%, 60%, 65%, 70%, 75%, 80%, 85%, or 90% to about 90%, 85%, 80%, 75%, 70%, 65%, 60%, 55%, 50%, 45%, 40%, 35%, 30%, 25%, 20%, 15%, 10%, 5%, or 3% of one or more oils. Oils may be used to solubilize, disperse, or carry materials that are not suitable for water or water soluble solvents. Suitable oils include silicones, hydrocarbons, esters, amides, ethers, and mixtures thereof. The oils may be volatile or nonvolatile.

Suitable silicone oils include polysiloxanes. Commercially available polysiloxanes include the polydimethylsiloxanes, which are also known as dimethicones, examples of which include the DM-Fluid series from Shin-Etsu, the Vicasif series sold by Momentive Performance Materials Inc., and the Dow Corning® 200 series sold by Dow Corning Corporation. Specific examples of suitable polydimethylsiloxanes include Dow Corning* 200 fluids (also sold as Xiameter” PMX-200 Silicone Fluids) having viscosities of 0.65, 1.5, 50, 100, 350, 10.000, 12,500 100,000, and 300,000 centistokes.

Suitable hydrocarbon oils include straight, branched, or cyclic alkanes and alkenes. The chain length may be selected based on desired functional characteristics such as volatility. Suitable volatile hydrocarbons may have between 5-20 carbon atoms or, alternately, between 8-16 carbon atoms. Other suitable oils include esters. The suitable esters typically contained at least 10 carbon atoms. These esters include esters with hydrocarbyl chains derived from fatty acids or alcohols (e.g., mono-esters, polyhydric alcohol esters, and di- and tri-carboxylic acid esters). The hydrocarbyl radicals of the esters hereof may include or have covalently bonded thereto other compatible functionalities, such as amides and alkoxy moieties (e.g., ethoxy or ether linkages, etc.).

Other suitable oils include amides. Amides include compounds having an amide functional group while being liquid at 25° C. and insoluble in water. Suitable amides include N-acetyl-N-butylaminopropionate, isopropyl N-lauroylsarcosinate, and N,N-diethyltoluamide. Other suitable amides are disclosed in U.S. Pat. No. 6,872,401. Other suitable oils include ethers. Suitable ethers include saturated and unsaturated fatty ethers of a polyhydric alcohol, and alkoxylated derivatives thereof. Exemplary ethers include C4. 20 alkyl ethers of polypropylene glycols, and di-Cs-3o alkyl ethers. Suitable examples of these materials include PPG-14 butyl ether, PPG-15 stearyl ether, dioctyl ether, dodecyl octyl ether, and mixtures thereof.

The transdermal delivery compositions disclosed herein can comprise an emulsifier. An emulsifier is particularly suitable when the composition is in the form of an emulsion or if immiscible materials are being combined. The topical therapeutic and/or cosmetic composition may comprise from about 0.05%, 0.1%, 0.2%, 0.3%, 0.5%, or 1% to about 20%, 10%, 5%, 3%, 2%, or 1% emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's, Emulsifiers and Detergents, 2010 Annual Ed., published by M. C. Publishing Co.

Other suitable emulsifiers are further described in the Personal Care Product Council's International Cosmetic Ingredient Dictionary and Handbook, Thirteenth Edition, 2006, under the functional category of “Surfactants—Emulsifying Agents.” Linear or branched type silicone emulsifiers may also be used. Particularly useful polyether modified silicones include KF-601 1, KF-6012, KF-6013, KF-6015, KF-6015, KF-6017, KF-6043, KF-6028, and F-6038 from Shin Etsu. Also particularly useful are the polyglycerolated linear or branched siloxane emulsifiers including KF-6100, KF-6104, and KF-6105 from Shin Etsu.

Emulsifiers also include emulsifying silicone elastomers. Suitable silicone elastomers may be in the powder form, or dispersed or solubilized in solvents such as volatile or nonvolatile silicones, or silicone compatible vehicles such as paraffinic hydrocarbons or esters. Suitable emulsifying silicone elastomers may include at least one polyalkyl ether or polyglycerolated unit.

Structuring agents may be used to increase viscosity, thicken, solidify, or provide solid or crystalline structure to the transdermal delivery compositions disclosed herein. Structuring agents are typically grouped based on solubility, dispersability, or phase compatibility. Examples of aqueous or water structuring agents include polymeric agents, natural or synthetic gums, polysaccharides, and the like. In one aspect, the topical therapeutic and/or cosmetic compositions may comprise from about 0.0001%, 0.001%, 0.01%, 0.05%, 0.1%, 0.5%, 1%, 2%, 3%, 5% to about 25%, 20%, 10%), 7%, 5%, 4%, or 2%, by weight of the composition, of one or more structuring agents.

Polysaccharides and gums may be suitable aqueous phase thickening agents. Suitable classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof. Silicone gums are another oil phase structuring agent. Another type of oily phase structuring agent includes silicone waxes. Silicone waxes may be referred to as alkyl silicone waxes which and are semi-solids or solids at room temperature. Other oil phase structuring agents may be one or more natural or synthetic waxes such as animal, vegetable, or mineral waxes.

The transdermal delivery compositions disclosed herein can comprise a gelling agent. The term “gelling agent” refers to materials used to thicken and stabilize liquid solutions, emulsions, and suspensions. They dissolve in the liquid phase as a colloid mixture that forms an internal structure giving the resulting gel an appearance of a solid matter, while being mostly composed of a liquid. Gelling agents are very similar to thickeners.

The transdermal delivery compositions disclosed herein can comprise a surfactant. A “surfactant” or “surface-active agent” refers to an organic compound that reduces the surface tension when dissolved in water or water solutions. In an emulsion, a surfactant will contain a hydrophilic portion and a lipophilic portion by which it functions to reduce the surface tension of the surfaces between immiscible phases. Functionally, surfactants include emulsifying agents, wetting agents, cleansing agents, foam boosters, and solubilizing agents.

A surfactant is any nonionic, anionic, cationic or zwitterionic (e.g., including, but not limited, betaines (e.g., cocamidopropyl betaine), detergents and amino acids) compound of moderate to high molecular weight (such as from about 100 to 300,000 Daltons) for which a significant portion of the molecule is hydrophilic and a significant portion is lipophilic.

The transdermal delivery compositions disclosed herein can be generally prepared by conventional methods such as known in the art of making compositions and topical compositions. Such methods typically involve mixing of ingredients in or more steps to a relatively uniform state, with or without heating, cooling, application of vacuum, and the like. Typically, emulsions are prepared by first mixing the aqueous phase materials separately from the fatty phase materials and then combining the two phases as appropriate to yield the desired continuous phase.

The transdermal delivery compositions disclosed herein are preferably prepared such as to optimize stability (physical stability, chemical stability, photostability, etc.) and/or delivery of active materials. The transdermal delivery compositions disclosed herein can be provided in a package sized to store a sufficient amount of the composition for a treatment period.

In some aspects, the transdermal delivery compositions disclosed herein can be prepared and/or administered, for example, as a solution, a gel, a cream, a lotion, an ointment, an emulsion, a suspension, a paste, an aerosol, an aerosol foam, an aerosol powder, a lotion, a liniment, an ointment, a tincture, a salve, a poultice, a spray, a dry power, or a combination thereof.

The term “solution” refers to a system at chemical equilibrium in which a solute (e.g., a therapeutic or cosmetic agent) is dissolved in a liquid solvent.

The term “gel” or “jelly” refers to solid, jelly-like materials made up of a substantially dilute crosslinked system, which exhibits no flow when in the steady-state. By weight, gels are mostly liquid, yet they behave like solids due to a three-dimensional crosslinked network within the liquid.

The term “cream” refers to topical preparations for application to the skin or mucous membranes such as those of the rectum or vagina. Creams are semisolid emulsions that are mixtures of oil and water. They are divided into two types: oil-in-water (OAV) creams that are composed of small droplets of oil dispersed in a continuous aqueous phase, and water-in-oil (W/O) creams that are composed of small droplets of water dispersed in a continuous oily phase.

The term “emulsion” refers to a mixture of two or more immiscible (unblendable) liquids. One liquid (the dispersed phase) is dispersed in the other (the continuous phase). Emulsions can be oil-in-water emulsions or water-in-oil emulsions.

The term “suspension” refers to a mixture in which fine particles are suspended in a fluid where they are supported by buoyancy; as well as a mixture in which fine particles are denser than the fluid and are not supported by buoyancy.

The term “paste” refers to a form consisting of a fatty base, water, and at least a solid substance in which a powder is suspended.

The term “aerosol” refers to a suspension of fine solid particles or liquid droplets in a gas.

The term “aerosol foam” refers to substance that is formed by trapping many gas bubbles in a liquid or solid.

The term “aerosol powder” refers to a type of dispensing system which creates an aerosol mist of solid particles.

The term “liniment” refers to a medicated topical preparation for application to the skin. Preparations of this type are also called balms or embrocation. Liniments are of a similar viscosity to lotions. Liniments are generally significantly less viscous than ointments or creams.

The term “lotion” refers to a low- to medium-viscosity topical preparation.

The term “ointment” refers to a viscous, homogeneous, semi-solid preparation used topically on a variety of body surfaces, such as the skin and the mucus membranes of the eye (an eye ointment), vagina, anus, and nose.

The term “tincture” refers to an alcoholic extract or solution of a non-volatile substance. To qualify as a tincture, the alcoholic extract is to have an ethanol percentage of at least 40-60%.

The term “salve” refers to a medicinal ointment used to soothe the head or other body surface.

The term “poultice” refers to a soft moist mass, often heated and medicated, that is spread on cloth over the skin to treat an aching, inflamed, or painful part of the body.

The term “spray” refers to a collection of liquid drops and the entrained surrounding gas.

In some specific aspects, the cosmetic or therapeutic compositions disclosed herein comprise inclusion bodies wherein the cosmetic and/or therapeutic agent in the inclusion bodies comprises, consists, or consists essentially of IL-10, KGF, EGF, VEGF, or combinations thereof.

### V. Transdermal Delivery System and Apparatus

The instant disclosure also provides a transdermal delivery system comprising a cosmetic composition comprising at least one cosmetic agent in inclusion body form. Also provided is a transdermal delivery system comprising a therapeutic composition comprising at least one therapeutic agent in inclusion body form. The transdermal delivery system can be, for example, a patch, a spray metered, a spray suspension, a swab, a sponge, a stick, a shampoo suspension, an aerosol metered, or an aerosol spray.

The terms “patch,” “transdermal patch,” or “skin patch” refer to a adhesive patch that is placed on the skin to deliver a specific dose of a therapeutic or cosmetic composition comprising at least one cosmetic or therapeutic agent in inclusion body form to and through the skin. Patches can provide controlled release of the therapeutic or cosmetic composition to the subject over an extended period of time.

The term “spray metered” refers to a device that helps deliver a specific amount of a therapeutic or cosmetic composition comprising at least one cosmetic or therapeutic agent in inclusion body form by supplying a short burst of liquid drops and the entrained surrounding gas.

The term “spray suspension” refers to a suspension of an active agent (e.g., a cosmetic agent or a therapeutic agent) in a liquid such that it can be sprayed onto a surface (such as skin) as a suspension of the active agent in a very small drops of liquid entrained in surrounding gas.

The term “swab” refers to a small piece of material, such as gauze or cotton, which is used to apply a therapeutic or cosmetic composition comprising at least one cosmetic or therapeutic agent in inclusion body form.

The term “sponge” refers to a mass of absorbent, porous plastics, rubber, cellulose, or other material, similar in absorbency used for bathing, cleaning, and other purposes.

The terms “stick” or “lipstick” refer to “stick-shaped” materials usually manufactured from beeswax or petroleum jelly that provide an occlusive surface and seal in moisture. The occlusive materials prevent moisture loss and maintain lip comfort, while flavorants, colorants, sunscreens and various agents can provide additional, specific benefits.

The term “shampoo” refers to any of various liquid or cream preparations of soap or detergent used to wash the hair and scalp. Shampoos containing dissolved or dispersed active agents, e.g., therapeutic or cosmetic agents, and can be used for transdermal delivery of said agents.

The term “shampoo suspension” refers to a shampoo containing a suspended active agent (e.g., a cosmetic agent or a therapeutic agent) in a shampoo for transdermal delivery of the agent during washing.

The term “aerosol metered” refers to a device that helps deliver a specific amount of a therapeutic composition or cosmetic composition comprising at least one cosmetic or therapeutic agent in inclusion body form by supplying a short burst of aerosolized medicine.

The term “aerosol spray” refers to a type of dispensing system which creates an aerosol mist of liquid particles.

The instant disclosure also provides a device or apparatus comprising a vessel joined to an applicator and a transdermal delivery system. Accordingly, in some aspects, the transdermal delivery composition (e.g., a therapeutic composition or a cosmetic composition comprising at least one cosmetic or therapeutic agent in inclusion body form disclosed herein) is incorporated into a device that facilitates application.

The apparatus generally comprises a vessel joined to an applicator, wherein a transdermal delivery composition of the instant disclosure (e.g., a therapeutic composition or a cosmetic composition comprising at least one cosmetic or therapeutic agent in inclusion body form) is incorporated in the vessel. Some devices, for example, facilitate delivery by encouraging vaporization of the mixture. These apparatus have a transdermal delivery composition of the present disclosure (e.g., a therapeutic composition or a cosmetic composition comprising at least one cosmetic or therapeutic agent in inclusion body form) incorporated in a vessel that is joined to an applicator such as a sprayer (e.g., a pump-driven sprayer). These aspects can also comprise a propellant for driving the incorporated transdermal delivery composition (e.g., a therapeutic composition or a cosmetic composition comprising at least one cosmetic or therapeutic agent in inclusion body form) out of the vessel. Other apparatus can be designed to allow for a more focused application. A device that facilitates a focused application of a transdermal delivery composition of the instant disclosure (e.g., a therapeutic composition or a cosmetic composition comprising at least one cosmetic or therapeutic agent in inclusion body form) can have a roll-on or swab-like applicator joined to the vessel that houses the transdermal delivery composition (e.g., a therapeutic composition or a cosmetic composition comprising at least one cosmetic or therapeutic agent in inclusion body form).

In some specific aspects, the transdermal delivery systems disclosed herein comprise a cosmetic or therapeutic composition comprising inclusion bodies wherein the cosmetic and/or therapeutic agent in the inclusion bodies comprises, consists, or consists essentially of IL-10, KGF, EGF, VEGF, or combinations thereof.

### VI. Diseases and Conditions and Methods of Treatment

The methods, compositions, delivery systems, and apparatuses disclosed in the instant application can be used in therapeutic (including prophylactic), and cosmetic applications to treat skin disorders. In addition, in some aspects, the methods, compositions, delivery systems, and apparatuses disclosed in the instant application can be used therapeutically (including prophylactically) to treat diseases other than skin disease. In some aspects, the methods, compositions, delivery systems, and apparatuses disclose in the instant application can be used to delivery therapeutic agents to locations distant from the skin surface via the bloodstream.

The terms “skin condition,” “skin disorder” and “skin disease” are used herein as referring to a physiological state (e.g., a pathological state) that can be prevented or treated by administration of a cosmetic or therapeutic agent in inclusion body form as described herein. The term includes, for example, skin conditions, disorders, or diseases associated with or caused by infection, inflammation, sun damage, or natural aging. Detailed examples of skin conditions and disorders that can be treated using methods, compositions, delivery systems, and apparatuses disclosed in the instant application are provided below.

Terms such as “treating” or “treatment” or “to treat” or “alleviating” or “to alleviate” refer to both (1) measures that cure, slow down, lessen symptoms of, and/or halt progression of a condition or disorder, for example, a skin condition or disorder, and (2) prophylactic or preventative measures that prevent and/or slow the development of a targeted condition or disorder, for example, a skin condition or disorder. Thus, those in need of treatment include those already with the condition or disorder; those prone to have the condition or disorder; and those in whom the condition or disorder is to be prevented.

In certain aspects, a subject is successfully “treated” according to the methods of the present disclosure if the subject shows, e.g., total, partial, or transient improvement of a condition or disorder, for example, a skin condition or disorder. For example, treatment or treating can include, but are not limited to: reduction in size and in thickness and hyperkeratinization; reduced pain; reduced itching; reduced inflammation adjacent to, but not the actual treatment area (redness and swelling away from the zone of topical application); reduction in the number of lesions; reduction in the occurrence of new lesions, resolution of lesions beyond the treated area (“field effect”), and reduction in rates of remission, e.g., due to increased immune surveillance.

The term “improve” as used herein, for example to refer to the improvement of a condition, e.g., a skin condition or disorder, after topical treatment with an therapeutic or cosmetic agent in inclusion body form, refers to any statistically significant improvement in a parameter measuring or quantitating the status of the skin condition or disorder (e.g., presence or absence and/or depth or length of wrinkles in skin aging). Accordingly, the term encompasses an improvement of about 10%, 20%, 30%, 40%, 50%, 60%, 70%, 80%, 90% or 100% in such parameter. The term improve also refers to at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, or at least about 180-fold or more improvement with respect to the untreated condition or disorder.

As used herein, the terms “prevent” and “preventing” include the prevention of the recurrence, spread, or onset of a disease or disorder, e.g., a skin condition or disorder. It is not intended that the methods, compositions, delivery systems, and apparatuses disclosed herein be limited to complete prevention. In some aspects, the onset is delayed, or the severity of the disease or disorder, for example, as skin condition or disorder, is reduced.

Many aspects are suitable for treatment of subjects either as a preventive measure (e.g., to avoid pain or skin disorders) or as a therapeutic measure to treat subjects already afflicted with skin disorders or who are suffering pain. In general, most drugs, chemicals, and cosmetic agents that can be incorporated into a pharmaceutical or cosmetic can be formulated into a transdermal delivery composition of the invention.

The aspects of the invention that follow are for exemplary purposes only, and one of skill in the art can readily appreciate the wide spread applicability of the transdermal delivery compositions disclosed herein (e.g., a therapeutic composition or a cosmetic composition comprising at least one cosmetic agent or therapeutic agent in inclusion body form) and the incorporation of other delivered agents (e.g., other therapeutic agents and/or cosmetic agents) into a transdermal delivery composition is straight forward.

In some aspects, the term skin disorder refers to a disease or condition that affects the health of a subject's skin. In some aspects, the term skin disorder also encompasses diseases or disorders affecting mucous membranes. In some aspects, the skin disorder is, for example, acne (including acne vulgaris, acne cystic, etc.), bed sores, rash, dry skin, dermal abrasions, dermatitis, sunburn, scars, hyperkeratosis, granuloma, skin ulceration, athlete's foot, canker sore, carbuncle, candidiasis, bacterial vaginitis, vaginosis, cellulitis, cold sores, dandruff, dermatitis (including, but not limited to, atopic dermatitis, contact dermatitis, serborrhoeic dermatitis, cradle cap, nummular dermatitis, perioral dermatitis, and dermatitis herpetiformis), eczema, erythrasma, erysipelas, erythema multiforme, furuncle, impetigo, infection (including, but not limited to, bacterial, viral, and fungal infections), vesicular bullous eruptions, cellulite, skin aging, skin wrinkles, hyperpigmentation, keratosis, skin blemish, dandruff, warts, photodamaged skin, chronic dermatoses, dermatitis, dryness, ichthyosis, etc.

In one embodiment, for example, a method of treatment or prevention of inflammation, pain, or human diseases, such as cancer, arthritis, and Alzheimer's disease, comprises using a transdermal delivery composition described herein (e.g., a therapeutic composition comprising at least one therapeutic agent in inclusion body form). By one approach, a transdermal delivery composition comprising a delivered agent (e.g., a therapeutic agent in inclusion body form) that is effective at reducing pain or inflammation is administered to a subject in need and the reduction in pain or inflammation is monitored. The transdermal delivery composition described herein (e.g., a therapeutic composition comprising at least one therapeutic agent in inclusion body form) is preferably applied to the skin at a region of inflammation or an area associated with pain or the particular condition and treatment is continued for a sufficient time to reduce inflammation, pain, or inhibit the progress of the disease.

In another method, an approach to reduce wrinkles and increase skin tightness and flexibility (collectively referred to as “restoring skin tone”) is provided. Accordingly, a transdermal delivery composition (e.g., a therapeutic composition or a cosmetic composition comprising at least one therapeutic agent and/or a cosmetic agent in inclusion body form) is provided and contacted with the skin of a subject in need of treatment.

Additionally, a method of reducing wrinkles, removing age spots, and increasing skin tightness and flexibility is provided. By this approach, a transdermal delivery composition comprising a therapeutic agent and/or a cosmetic agent in inclusion body form disclosed herein is provided to a subject in need, the subject is contacted with the transdermal delivery composition, and treatment is continued for a time sufficient to restore a desired skin tone (e.g., reduce wrinkles, age spots, or restore skin brightness, tightness and flexibility).

Specific definitions of some skin conditions and disorders that can be treated using the methods, compositions, delivery systems, and apparatuses disclosed in the instant application are provided below.

The term “carbuncle” refers to an abscess larger than a boil, usually with one or more openings draining pus onto the skin. It is usually caused by bacterial infection, most commonly Staphylococcus aureus. The term “cellulitis” refers to a diffuse infection of connective tissue with severe inflammation of dermal and subcutaneous layers of the skin. Cellulitis is caused by a type of bacteria entering the skin, usually by way of a cut, abrasion or break in the skin. Group A Streptococcus and Staphylococcus are the most common of these bacteria. The term “dermatitis” refers to any inflammation of the skin (e.g. rashes, etc.). The term “dermatophytosis” refers to a group of mycosis infections of the skin caused by parasitic fungi (dermatophytes).

The term “ecthyma” refers to a variation of impetigo, presenting at a deeper level of tissue. It is usually associated with Staphylococcus. The term “eczematous dermatitis,” or “eczema” as it is commonly called, is a type of allergic condition that affects the upper layers of the skin. The condition is characterized by persistent and recurring skin rashes with redness, itching, dryness and skin edema. The term “erysipelas” refers to an acute streptococcus bacterial infection of the dermis, resulting in inflammation and characteristically extending into underlying fat tissue.

The term “erythema multiforme” refers to a skin condition of unknown etiology, possibly mediated by deposition of immune complex in the superficial microvasculature of the skin and oral mucous membrane that usually follows an antecedent infection or drug exposure. The mild form usually presents with mildly itchy, pink-red blotches, symmetrically arranged and starting on the extremities. It often takes on the classical “target lesion” appearance, with a pink-red ring around a pale center. The term “erythrasma” refers to a skin disease that can result in pink patches, which can turn into brown scales. It is caused by the bacterium Corynebacterium minutissimum. The term “erythroderma” (also known as “Exfoliative dermatitis,” “Dermatitis exfoliativa,” and “Red man syndrome”) refers to an inflammatory skin disease with erythema and scaling that affects nearly the entire cutaneous surface.

The term “folliculitis” refers to the inflammation of one or more hair follicles. The condition may occur anywhere on the skin. The term “furuncle” (or “boil”) refers to a skin disease caused by the infection of hair follicles, resulting in the localized accumulation of pus and dead tissue. The term “impetigo” refers to a superficial bacterial skin infection. It is primarily caused by Staphylococcus aureus, and sometimes by Streptococcus pyogenes.

The term “staphylococcal scalded skin syndrome” also known as Pemphigus neonatorum or Ritter's disease, refers to a dermatological condition caused by Staphylococcus aureus. The term “trichomoniasis” refers to an infection that is a common cause of vaginitis. It is caused by the single-celled protozoan parasite Trichomonas vaginalis. The term “vaginosis” (e.g., bacterial vaginosis) refers to a vaginal infection (vaginitis). It is caused by an imbalance of naturally occurring bacterial flora or the presence of yeast (candidiasis) or Trichomonas vaginalis (trichomoniasis). The term “vesicular bullous eruptions” refers to blistering illnesses caused by bacteria, viruses, systemic illness, or sun or heat exposure.

As used herein, the term “skin aging” refers to a human skin tissue condition resulting from the expression or repression of genes, environmental factors (e.g., sun exposure, UVA and/or UVB exposure, smoking), intrinsic factors (e.g. endogenous free radical production or cellular senescence) or interactions there between that produces one or more of fine lines and/or wrinkles, dry skin, inflamed skin, rough skin, sallow skin, telangectasia, sagging skin, enlarged pores, and combinations thereof. As used herein, the term “intrinsic aging skin condition” refers to a skin aging condition that derives, in whole or part, from chronological aging of the skin. As used herein, the term “photo-aging skin condition” refers to a skin aging condition that derives, in whole or part, from exposure to sunlight and/or ultraviolet light (e.g., UVR, UVA, UVB, and/or UVC).

As used herein, the term “skin cancer” is used to refer to malignant and premalignant skin cancers. As such, the term “skin cancer” is inclusive of melanoma and non-melanoma skin cancers, actinic keratoses, basal cell carcinomas, squamous cell carcinoma-in-situ or Bowen's disease, melanoma in-situ, and other unresectable carcinomas. The term “skin cancer” refers to both primary and secondary cancers of the skin. In this regard, the term is inclusive of metastatic lesions caused by a primary skin cancer or another cancer that metastasizes to the skin. Further, the term includes the following nonlimiting examples: cutaneous T-cell lymphoma, extramammary Paget's disease, lentigo maligna, cutaneous melanoma metastases, and cutaneous leishmaniasis.

In some specific aspects, diseases and/or conditions disclosed herein can be treated by the transdermal administration of inclusion bodies comprising, consisting of, or consisting essentially of IL-10, KGF, EGF, VEGF, or combinations thereof. In some specific aspects, inclusion bodies comprising, consisting of, or consisting essentially of IL-10, KGF, EGF, VEGF, or combinations thereof can be administered transdermally for cosmetic purposes.

### VII. Inclusion Body Production and Purification

Therapeutic agents and cosmetics agents in inclusion body form can be produced by numerous methods known in the art without undue experimentation.

In some aspects, inclusion body formation can be promoted by the genetic fusion (or chemical conjugation) of a therapeutic or cosmetic protein of interest to an inclusion-body inducing polypeptide. In some aspects, the inclusion-body inducing polypeptide is a viral protein. In particular aspects, the viral protein is a capsid protein. In some aspects, the inclusion body-inducing polypeptide comprises the VP1 pentamer-forming capsid protein of Foot and Mouth Disease Virus (FMDV) or a fragment thereof. Other IB-inducing proteins are known in the art. Thus, virtually any therapeutic or cosmetic protein selected for expression could be directed to deposit as an inclusion body and subsequently used as disclosed herein.

In some specific aspects, the inclusion bodies disclosed herein comprise, consist of, or consist essentially of IL-10, KGF, EGF, VEGF, or combinations thereof.

As much as 20-40% of human gene constructs can, under normal cell culture conditions, express as inclusion bodies in E. coli (Stevens (2000) Structure Fold. Des. 8:R177-185). In addition, IB formation can be triggered by multiple factors such as higher induction temperatures, cell cultivation without pH control, osmolarity changes, induction modality, choice of promoter, cell density, culture medium, or—in general—any factor affecting the total expression rate of the system.

The inclusion bodies of the instant disclosure can be obtained by conventional methods which generally comprise introducing the sequence of nucleic acids encoding the therapeutic or cosmetic protein of interest in a suitable expression system which can produce IBs and culturing it under conditions suitable for the production of said IBs.

The term “bacteria” as used herein includes eubacteria and archaebacteria. In certain aspects, eubacteria, including gram-positive and gram-negative bacteria, are used in the methods described herein. In one aspect, gram-negative bacteria are used, e.g. Enterobacteriaceae. Examples of bacteria belonging to Enterobacteriaceae include Escherichia, Enterobacter, Erwinia, Klebsiella, Proteus, Salmonella, Serratia, and Shigella.

In one aspect, E. coli is used. MC4100, DnaK, and BL21 are suitable E. coli strains used in some aspects. Other suitable E. coli strains include E. coli LG 1522 (ATCC No.: BAA-1907™), E. coli AMC 198 (ATCC No.: CRM-11229™), E. coli Crooks (ATCC No.: CRM-8739™), DH5α, BL26, HB101, JM107, D21, JM103, AB 1157, and in general any of the strains available at the Yale University Coli Genetic Stock Center or other repositories (Maloy & Hughes (2007) Methods Enzymol. 421:3-8).

In some aspects, gram-positive bacteria are used, e.g. Lactobacillales. Examples of bacteria belonging to the order Lactobacillales include Lactococcus, Lactobacillus, Pediococcus, Oenococcus, Leuconostoc, Enterococcus, and Streptococcus (see, e.g., Ljungh & Wadstrom (2009) “Lactobacillus Molecular Biology: From Genomics to Probiotics,” Horizon Scientific Press, ISBN 1904455417; Charalampopoulos & Rastall (2009) “Prebiotics and Probiotics Science and Technology,” Springer ISBN 0387790578). In some specific aspects, the bacteria is a strain of Lactococcus lactis. In some aspects, the Lactococcus lactis strain is protease deficient. In some aspects, the Lactococcus lactis strain is L. lactis NZ900 HtraA-ClpP-.

These examples are illustrative rather than limiting. Mutant cells of any of the above-mentioned bacteria can also be employed. It is, of course, necessary to select the appropriate bacteria taking into consideration the replicability of the replicon in the cells of a bacterium. For example, E. coli, Serratia, or Salmonella species can be suitably used as the host when well-known plasmids such as pBR322, pBR325, pACYC177, or pKN410 or other commercially available vectors are used to supply the replicon. In some aspects, fungi can be used, e.g., Geotrichum candidum, Kluveromyces marxianus, and Pichia fermentans.

As used herein, the expressions “cell,” “cell line,” “strain,” and “cell culture” are used interchangeably and all such designations include progeny. Thus, when a nucleic acid encoding a therapeutic or cosmetic protein is introduced in a “cell” or “cell line,” the term includes the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

The introduction of the sequence encoding the therapeutic or cosmetic protein in the microorganisms and cell lines is carried out by means of conventional methods. In brief, expression vectors capable of autonomous replication and protein expression relative to the host cell genome are introduced into the host cell. Construction of appropriate expression vectors is well known in the art. See, e.g., Sambrook et al. (2001) “Molecular Cloning, A Laboratory Manual”, Cold Spring Harbor Laboratory Press (Cold Spring Harbor, N.Y.); Ausubel et al. (1994) “Current Protocols in Molecular Biology” (New York: Greene Publishing Associates and Wiley-Interscience); and Baneyx (1999) Current Opinion in Biotechnology 10:411-421.

Appropriate prokaryotic cells, including bacteria, and expression vectors are available commercially through, for example, the American Type Culture Collection (ATCC, Rockville, Md.). Methods for the large scale growth of prokaryotic cells, and especially bacterial cell culture are well known in the art and these methods can be used in the context of the instant disclosure. In some aspects, the pTVP IGFP (Apr), pReceiver-MO2, and pReceiver-B01 vectors (Genecopoeia, Rockville, Md.) can be used.

For example, prokaryotic host cells can be transfected with expression or cloning vectors encoding the recombinant therapeutic or cosmetic protein of interest and cultured in conventional nutrient media modified as appropriate for inducing promoters, selecting transformants, or amplifying the genes encoding the desired sequences. The nucleic acid encoding the therapeutic or cosmetic protein of interest can be RNA, cDNA, or genomic DNA from any source, provided it encodes the polypeptide(s) of interest. Methods are well known for selecting the appropriate nucleic acid for expression of polypeptides and proteins (including variants thereof) in microbial hosts. Nucleic acid molecules encoding the therapeutic or cosmetic protein of interest are prepared by a variety of methods known in the art. For example, a DNA encoding Hsp70 can be isolated and sequenced, e.g., by using oligonucleotide probes that are capable of binding specifically to the gene encoding Hsp70. Similarly, a DNA encoding IL-10, EGF, KGF, VEGF, or a fragment, variant, or derivative thereof can be isolated and sequenced, e.g., by using oligonucleotide probes that are capable of binding specifically to the gene encoding IL-10, EGF, KGF, or VEGF, respectively.

The nucleic acid (e.g., cDNA or genomic DNA) encoding the therapeutic or cosmetic protein can be inserted into a replicable vector for expression in the microorganism under the control of a promoter. Many vectors are available for this purpose, and selection of the appropriate vector will depend mainly on the size of the nucleic acid to be inserted into the vector and the particular host cell to be transformed with the vector. Each vector contains various components depending on the particular host cell with which it is compatible. Depending on the particular type of host, the vector components generally include, but are not limited to, one or more of the following: a signal sequence, an origin of replication, one or more marker genes, a promoter, and a transcription termination sequence.

In some specific aspects, the nucleic acid encodes IL-10, EGF, KFG, VEGF (including fragments, variants, and derivatives thereof, such as fusion proteins, or constructs comprising a heterologous moiety) or a combination thereof. In some specific aspects, the therapeutic or cosmetic protein in the inclusion body can be encoded by a single nucleic acid. In other aspects, the therapeutic or cosmetic protein in the inclusion body can be encoded by multiple nucleic acids (e.g., a protein could comprise several subunits, each one of them could be encoded by a different nucleic acid, and each one of the different nucleic acids could be inserted in the same vector or in different vectors). In some aspects, the nucleic acids encoding the therapeutic or cosmetic proteins disclosed herein have been codon optimized.

In general, plasmid vectors containing replicon and control sequences that are derived from species compatible with the host cell are used in connection with microbial hosts. The vector ordinarily carries a replication site, as well as marking sequences that are capable of providing phenotypic selection in transformed cells.

(i) Signal Sequence:

Therapeutic and cosmetic proteins can be produced recombinantly not only directly, but also as a fusion polypeptide with a heterologous polypeptide, which is typically a signal sequence or other polypeptide having a specific cleavage site at the N-terminus of the mature protein or polypeptide. The signal sequence selected typically is one that is recognized and processed (i.e., cleaved by a signal peptidase) by the host cell. For prokaryotic host cells that do not recognize and process a heterologous polypeptide signal sequence, the signal sequence can be substituted by a prokaryotic signal sequence selected, for example, from the group of the alkaline phosphatase, penicillinase, 1 pp, or heat-stable enterotoxin II leaders.

(ii) Origin of Replication Component:

Expression vectors contain a nucleic acid sequence that enables the vector to replicate in one or more selected host cells. Such sequences are well known in the art for a variety of microbes.

(iii) Selection Gene Component:

Expression vectors generally contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies other than those caused by the presence of the genetic marker(s), or (c) supply critical nutrients not available from complex media, e.g., the gene encoding D-alanine racemase for Bacilli.

One example of a selection scheme utilizes a drug to arrest growth of a host cell. In this case, those cells that are successfully transformed with the nucleic acid of interest produce a polypeptide conferring drug resistance and thus survive the selection regimen. Examples of such dominant selection use the drugs neomycin (Southern & Berg (1982) J. Mol. Appl. Genet. 1: 327-341), mycophenolic acid (Mulligan & Berg (1980) Science 209:1422-27) or hygromycin (Sugden et al. (1985) Mol. Cell. Biol. 5:410-413). The three examples given above employ bacterial genes under eukaryotic control to convey resistance to the appropriate drug G418 or neomycin (geneticin), ×gpt (mycophenolic acid), or hygromycin, respectively.

(iv) Promoter Component:

The expression vector for producing the recombinant therapeutic or cosmetic protein of interest contains a suitable promoter that is recognized by the host organism and is operably linked to the nucleic acid encoding the therapeutic or cosmetic protein of interest. Promoters suitable for use with prokaryotic hosts include the beta-lactamase and lactose promoter systems (Chang et al. (1978) Nature 275:617-624; Goeddel et al. (1979) Nature 281:544-48), the arabinose promoter system (Guzman et al. (1992) J. Bacteriol. 174: 7716-7728), alkaline phosphatase, a tryptophan (trp) promoter system (Goeddel et al. (1980) Nucleic Acids Res. 8: 4057-74, and European Patent EP 36776) and hybrid promoters such as the tac promoter (De Boer et al. (1983) Proc. Natl. Acad. Sci. USA 80: 21-25). In one aspect, the recombinant genes can be expressed under the control of an isopropyl beta-D-1-thiogalactopyranoside (IPTG) inducible trc (trp-lac) promoter (Egon et al. (1983) Gene 25:167-178). However, other known bacterial promoters are suitable. Their nucleotide sequences have been published, thereby enabling a skilled worker operably to ligate them to DNA encoding the polypeptide of interest (Siebenlist et al. (1980) Cell 20:269-81) using linkers or adaptors to supply any required restriction sites. See also, e.g., Sambrook et al., supra; and Ausubel et al., supra.

Promoters for use in bacterial systems also generally contain a Shine-Dalgarno (S.D.) sequence operably linked to the DNA encoding the therapeutic or cosmetic protein of interest. The promoter can be removed from the bacterial source DNA by restriction enzyme digestion and inserted into the vector containing the desired DNA.

(v) Construction and Analysis of Vectors:

Construction of suitable vectors containing one or more of the above-listed components employs standard ligation techniques. Isolated plasmids or DNA fragments are cleaved, tailored, and re-ligated in the form desired to generate the plasmids required. For analysis to confirm correct sequences in plasmids constructed, successful transformants are selected by antibiotic resistance. Plasmids from the transformants are prepared, analyzed by restriction endonuclease digestion, and/or sequenced by the method of Sanger et al. (Sanger et al. (1977) Proc. Natl. Acad. Sci. USA 74:5463-5467) or Messing et al. (Messing et al. (1981) Nucleic Acids Res. 9:309-21), or by the method of Maxam & Gilbert (Maxam & Gilbert (1980) Methods in Enzymology 65:499-560). See also, e.g., Sambrook et al., supra; and Ausubel et al., supra.

The nucleic acid encoding the recombinant therapeutic or cosmetic protein of interest can then be inserted into the host cells. Typically, this is accomplished by transforming the host cells with the above-described expression vectors and culturing in conventional nutrient media modified as appropriate for inducing the various promoters.

(vi) Culturing the Host Cells:

As previously discussed, suitable cells are well known in the art. Host cells that express the recombinant therapeutic or cosmetic protein abundantly in the form of inclusion bodies or in the periplasmic or intracellular space are typically used. Prokaryotic cells used to produce the therapeutic protein are grown in media known in the art and suitable for culture of the selected host cells, including the media generally described by Sambrook et al., Molecular Cloning, A Laboratory Manual, Cold Spring Harbor Laboratory Press (Cold Spring Harbor, N.Y.) (2001). Media that are suitable for bacteria include, but are not limited to, Luria-Bertani (LB) broth, AP5 medium, nutrient broth, Neidhardt's minimal medium, and C.R.A.P. minimal or complete medium, plus necessary nutrient supplements. In certain aspects, the media also contains a selection agent, chosen based on the construction of the expression vector, to selectively permit growth of prokaryotic cells containing the expression vector. For example, ampicillin is added to media for growth of cells expressing ampicillin resistant gene. Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol, and dithiothreitol.

The prokaryotic host cells can be cultured at suitable temperatures. For E. coli growth, for example, the temperature ranges from, e.g., about 20° C. to about 39° C., or from about 25° C. to about 37° C., or at about 30° C. If the promoter is an inducible promoter, for induction to occur, typically the cells can be cultured until a certain optical density is achieved, e.g., a A₅₅₀ of about 200 using a high cell density process, at which point induction is initiated (e.g., by addition of an inducer, by depletion of a medium component, etc.), to induce expression of the gene encoding the therapeutic or cosmetic protein of interest.

Any necessary supplements can also be included at appropriate concentrations that would be known to those skilled in the art, introduced alone or as a mixture with another supplement or medium such as a complex nitrogen source. The pH of the medium can be any pH from about 5-9, depending mainly on the host organism. For E. coli, the optimal pH is, e.g., from about 6.8 to about 7.4, or about 7.0.

IBs can be isolated from host cells expressing the therapeutic or cosmetic protein by any of a number of art standard techniques. For example, the insoluble recombinant therapeutic or cosmetic protein is isolated in a suitable isolation buffer by exposing the cells to a buffer of suitable ionic strength to solubilize most host proteins, but in which the subject therapeutic or cosmetic protein is substantially insoluble, or disrupting the cells so as to release the inclusion bodies from the periplasmic or intracellular space and make them available for recovery by, for example, centrifugation. This technique is well known and is described in, for example, U.S. Pat. No. 4,511,503. Kleid et al., disclose purification of IBs by homogenization followed by centrifugation (Kleid et al. (1984) Soc. Industr. Microbiol. 23:217-235). See also, e.g., Fischer et al. (1993) Biotechnology and Bioengineering 41:3-13.

U.S. Pat. No. 5,410,026 describes a typical method for recovering protein from IBs and is summarized as follows. The prokaryotic cells are suspended in a suitable buffer. Typically the buffer consists of a buffering agent suitable for buffering between pH 5 to 9, or about 6 to 8 and a salt. Any suitable salt, including NaCl, is useful to maintain a sufficient ionic strength in the buffered solution. Typically, an ionic strength of about 0.01 to 2 M, or 0.1 to 0.2 M is employed. The cells, while suspended in this buffer are disrupted or lysed using techniques commonly employed such as, for example, mechanical methods, e.g. homogeneizer (Manton-Gaulin press, Microfluidizer, or Niro-Soavi), a French press, a bead mill, or a sonic disruptor (probe or bath), or by chemical or enzymatic methods.

Examples of chemical or enzymatic methods of cell disruption include spheroplasting, which entails the use of lysozyme to lyse the bacterial wall (Neu & Heppel (1964) Biochem. Biophys. Res. Comm. 17:215-19), and osmotic shock, which involves treatment of viable cells with a solution of high tonicity and with a cold-water wash of low tonicity to release the polypeptides (Neu & Heppel (1965) J. Biol. Chem. 240:3685-3692). Sonication is generally used for disruption of bacteria contained in analytical scale volumes of fermentation broth. At larger scales, high pressure homogenization is typically used.

After the cells are disrupted, the suspension is typically centrifuged at low speed, generally around 500 to 25,000×g, e.g., in one aspect about 15,000×g is used, in a standard centrifuge for a time sufficient to pellet substantially all of the insoluble protein.

Such times can be simply determined and depend on the volume being centrifuged as well as the centrifuge design. Typically about 10 minutes to 0.5 hours is sufficient to pellet the IBs. In one aspect, the suspension is centrifuged at 15,000×g for 15 minutes. The resulting pellet contains substantially all of the IBs.

If the cell disruption process is not complete, the pellet may also contain intact cells or broken cell fragments. Completeness of cell disruption can be assayed by resuspending the pellet in a small amount of the same buffer solution and examining the suspension with a phase contrast microscope. The presence of broken cell fragments or whole cells indicates that further sonication or other means of disruption is necessary to remove the fragments or cells and other contaminants. After such further disruption, if required, the suspension can be again centrifuged and the pellet recovered, resuspended and reexamined. The process can be repeated until visual examination reveals the absence of broken cell fragments in the pelleted material or until further treatment fails to reduce the size of the resulting pellet.

The above described process for recombinant production of inclusion bodies containing therapeutic or cosmetic agents can be employed whether the IBs are intracellular or in the periplasmic space. In one aspect, the conditions given herein for producing and isolating IBs are directed to IBs containing GFP fused to the amino terminus of VP1 and to IBs containing the Hsp70 chaperon. However, the processes and procedures are applicable to recombinant proteins in general with minor modifications. Accordingly, the same processes, methods, and conditions disclosed herein are generally applicable to the production, isolation, and characterization (e.g., biophysical and/or pharmacological) of IBs comprising cosmetically and/or therapeutically effective polypeptides wherein the polypeptide(s) comprise, consist, or consist essentially of IL-10 and/or EGF and/or KGF and/or VEGF and/or fragments, variants, or derivatives thereof.

In certain aspects, the processes and procedures are applicable to manufacturing or industrial scale production and purification of IBS containing a therapeutic or cosmetic protein.

It is known in the art that insoluble therapeutic or cosmetic proteins in IBs can be recovered in biologically active forms by solubilizing or diluting the IBs and refolding the protein (see, e.g., Burgess (2009) Methods in Enzymology 463:259-282; Cabrita & Bottomley (2004) Biotechnology Annual Review 10:31-50). These solubilized and refolded proteins can then be administered for therapeutic or cosmetic uses. The methods of topical administration of therapeutic and cosmetic proteins disclosed herein differ from methods known in the art in that the therapeutic or cosmetic proteins of interest are administered in IB form, and the IB penetrate the skin. Thus, expensive and time consuming solubilization and refolding steps to obtain a soluble and physiologically active protein are not necessary.

The synthesis of the therapeutic or cosmetic peptides can be carried out according to conventional methods known in the art, such as for example the adaptation of solid-phase peptide synthesis methods (Stewart J. M. and Young J. D. (1984) Solid Phase Peptide Synthesis, 2nd edition, Pierce Chemical Company, Rockford, Ill.; Bodanzsky M. and Bodanzsky A. (1984) The practice of Peptide Synthesis, Springer Verlag, New York; Lloyd-Williams et al. (1997) Chemical Approaches to the Synthesis of Peptides and Proteins. CRC, Boca Raton (Fla., USA)), solution synthesis, a combination of solid-phase synthesis and solution synthesis methods or enzymatic methods (Kullmann W (1980) J. Biol. Chem. 255, 8234-8238). These peptides can be obtained by the fermentation of a bacterial strain that is modified or unmodified by genetic engineering with the aim of producing the desired sequences.

All publications such a textbooks, journal articles, Genbank or other sequence database entries, published applications and patent applications mentioned in this specification are herein incorporated by reference to the same extent as if each individual publication or patent was specifically and individually indicated to be incorporated by reference.

### EXAMPLES

### Example 1

### GFP Inclusion Bodies can Penetrate Deeply into Model Epidermis Samples

### 1. Goal:

The goal of this study was to evaluate the potential of inclusion body samples to penetrate the epidermis. Inclusion bodies from a generally soluble protein were generated by fusing the protein to an inclusion body-inducing peptide. Inclusion body penetration was evaluated using STRATICELL® RHE-EPI/001, a Reconstituted Human Epidermis (RHE) model system widely used in the art as a surrogate of human skin for the development of skin therapies and cosmetics.

### 2. Materials and Methods

2.1 Products Evaluated:

Three samples were evaluated: (1) a positive control sample comprising soluble Green Fluorescent Protein (GFP) (50 μg in 50 μl of Tris buffer), designated M0037 GFP; (2) a sample comprising GFP bacterial inclusion bodies (50 g in pellet form), designated M0037 CI; and (3) a negative control (culture medium), designated M0037 CTRL.

2.2 Materials Used:

Reconstituted human epidermis (RHE/EPI/001) inserts; growth culture medium and maintenance culture medium, provided by Straticell; Phosphate Buffered Saline (PBS); scalpels, cryogenic vial grippers, vessels to manipulate the samples; isopentane (2-methylbutane); dry ice/liquid nitrogen; Optimum Cutting Temperature (OCT) compound; cryo molds, standard size; gelatinized sample holders and cover slips; mounting medium for fluorescence microscopy.

2.3 Equipment:

General equipment for cell cultures (CO₂ incubators, laminar flow cabinets, microscopes, centrifuges), Leica CM3050S cryostat, Olympus BX62 automated fluorescence microscope coupled to an Olympus DP70 digital image capture system, Leica stereoscopic fluorescence microscope.

2.4 Methods

2.4.1 Reconstituted Skin and Product Application:

RHE/EPI inserts were processed strictly following the instructions provided by STRATICELL®. After a 24 hour stabilization period at 37° C., in ambient 37° C., 5% CO₂ and humidity, the sample products were applied as follows:
M0037 GFP: 50 μg of GFP in 50 μL of Tris buffer plus 25 μL of maintenance medium.
M0037 CI: 50 μg of CI plus 75 μL of maintenance medium.
M0037 CTRL: 75 μl of maintenance medium.

After all products were applied, samples were incubated for 24 hours at 37° C., 5% CO₂. After the incubation period, samples were processed for freezing.

2.4.2 Mounting and Freezing

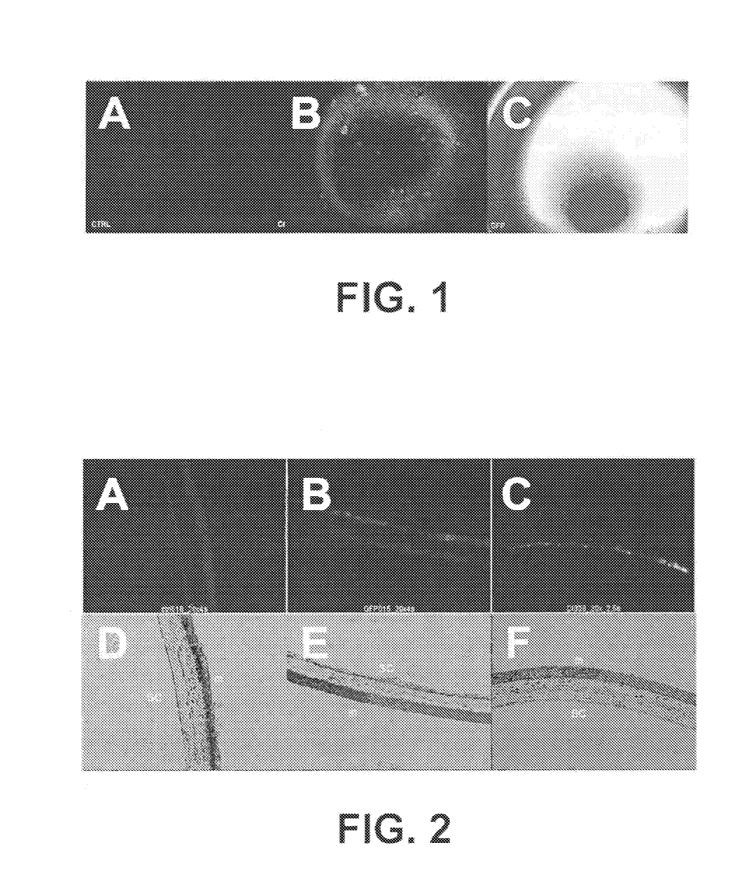

After the 24 hour incubation period, and before sample processing for freezing, the samples were visually evaluated using fluorescence microscopy (FIG. 1). From that point, samples were processed using the snap-freezing method (ultrafast freezing) to ensure maximum integrity and reduce the possibility of sample loss. The snap-freezing protocol followed is detailed below.

The SNAP-FREEZING procedure for preparation and freezing of samples was applied as follows. Isopentane was cooled by suspending the container in liquid nitrogen or dry ice. Isopentane was considered sufficiently cold when beads were formed and the solution looked dense and cloudy. A thin layer of OCT was deposited over a cryo mold that had previously been labeled and/or identified. The incubation solution was withdrawn from the reconstituted skin inserts. The membrane was then removed from the insert with the help of a scalpel, and the reconstituted skin sample was deposited on a Petri dish containing PBS at 4° C. Each one of the samples was lightly dried on a piece of filter paper (carefully avoiding any possible paper depositions on the epidermis) just before being introduced in the OCT medium. Each insert was divided in two in order to provide a duplicate sample. The reconstituted skin sample was positioned on the OCT layer on the cryo mold, orienting it so the different skin layers were on the base of the mold (i.e., the epidermis was oriented towards one of the edges). It was important to position the tissue correctly on the cryo mold to obtain optimal histological cuts using the cryostat. The deposited tissue fragment was covered with OCT. A scalpel or the tip of a pipette were used to finely position the tissue and to prevent the formation of air bubbles. The cryo mold was deposition in the precooled isopentane using a pair of tweezers. Once the sample was frozen, the cryo mold was kept in dry ice while the remaining samples were processed. Samples were stored in a freezer at −80° C. For transportation, liquid nitrogen or dry ice was used.

2.4.3 Preparation of Slices and Mounting

Once the samples were frozen, 20 μm thick slices were obtained using a LEICA CM3050 S cryostat. The slices were deposited onto gelatinized sample holders. Afterwards, a mounting medium for fluorescence detection (Fluoromount) was used to semipermanently mount the samples. Samples were stored at 4° C., away from the light.

2.4.4 Observation and Image Capture

Observations were conducted using an automated microscope (Olympus BX61) coupled to an image capture digital system (Olympus DP70) using a 20× plan apochromatic lens. Image names were codified, with the names indicating capture number (correlative number), magnification, and in some cases exposure time. In addition to the fluorescence image, a transmitted light image was captured for each sample to facilitate location of the fluorescent signal.

The capture of fluorescence images was conducted in manual mode instead of using automated mode, adjusting the exposure time between 3 and 4 seconds. Thus, the fluorescence intensity among samples was comparable. In some of the soluble GFP or control sample images, and always indicating it in the name of the image, the exposure was increased up to 30 seconds.

### 3. Long Term Sample Storage

After concluding the experiments, samples were kept for long term storage (up to two years) at −80° C.

### 4. Results

### 4.1. Fluorescence Comparison for the Three Samples

Panels A, B, and C in FIG. 2 show the relative fluorescence intensity of the three samples, i.e., control (CTRL), soluble GFP (GFP), and GFP inclusion bodies (CI). The magnification was 20× in each case. The exposure time was 4 seconds for the control and soluble GFP samples, and 2.5 seconds for the inclusion bodies sample. Since the exposure time was the same for each one of the samples, the differences in fluorescence intensity shown in the images corresponds to the differences in real intensity. Panels D, E, and F in FIG. 2 shows transmission microscopy images corresponding to the samples in Panels A, B, and C, respectively. Each one of the transmission images indicated the location of the membrane used to grow the artificial epidermis model (denoted by the letter “m”) and the location of the stratum corneum, i.e., the outermost layer of the epidermis (indicated by “SC”).

### 4.2. M0037-CI Sample (Inclusion Bodies)

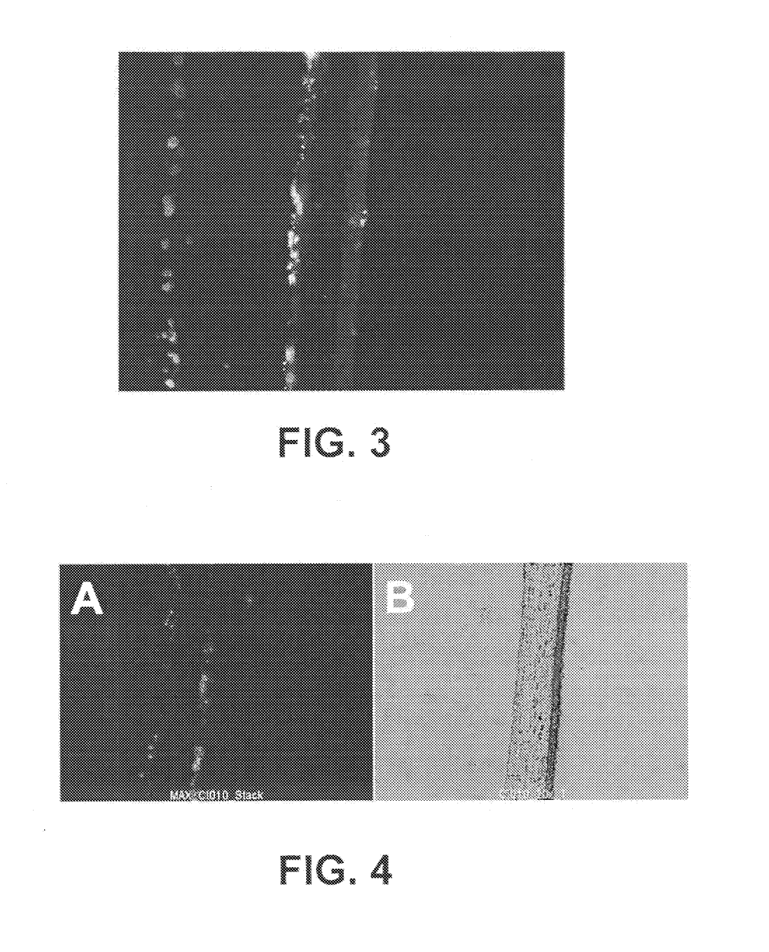

FIG. 3 and FIG. 4 provide several examples of fluorescence images obtained from the M0037-CI sample, i.e., GFP in inclusion body form. In most observations, the fluorescence was located in fluorescence aggregates located in the stratum corneum (SC) or in the outer layer of the epidermis. Since no additional staining or labeling was conducted, it was difficult to determine whether the inclusion bodies were localized in the stratum corneum or they had penetrated deeper in the epidermis. This level of penetration will be determined using a combination of stains (H&E, Col7, etc.) and observations via confocal microscopy. In several M0037-CI samples some SC fragments had begun to detach (see. e.g., FIG. 3 and FIG. 4). Accordingly, in those cases it was difficult to determine whether the inclusion bodies were mostly in the SC or they had penetrated deeply in the epidermis.

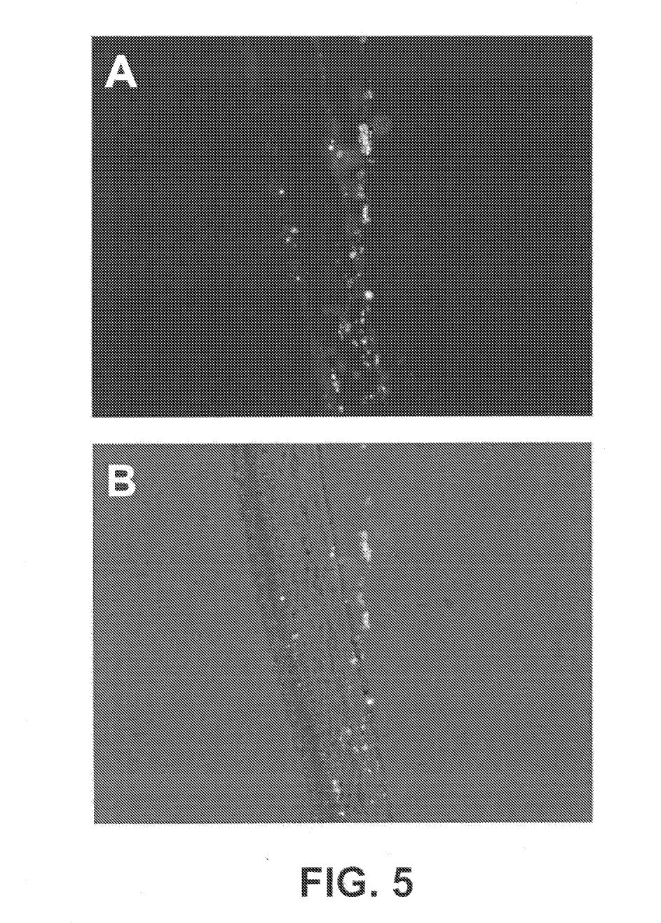

In some of the slices, labeling was observed deep in the epidermis. Since the slices had a thickness of 20 μm, conventional microscopy did not provide a completely clear view of the field. FIG. 5 shows a reconstruction corresponding to the maximum intensity of the 3 images of the same field taken at different focal plains.

### 4.3 M0037-GFP Sample (Soluble GFP)

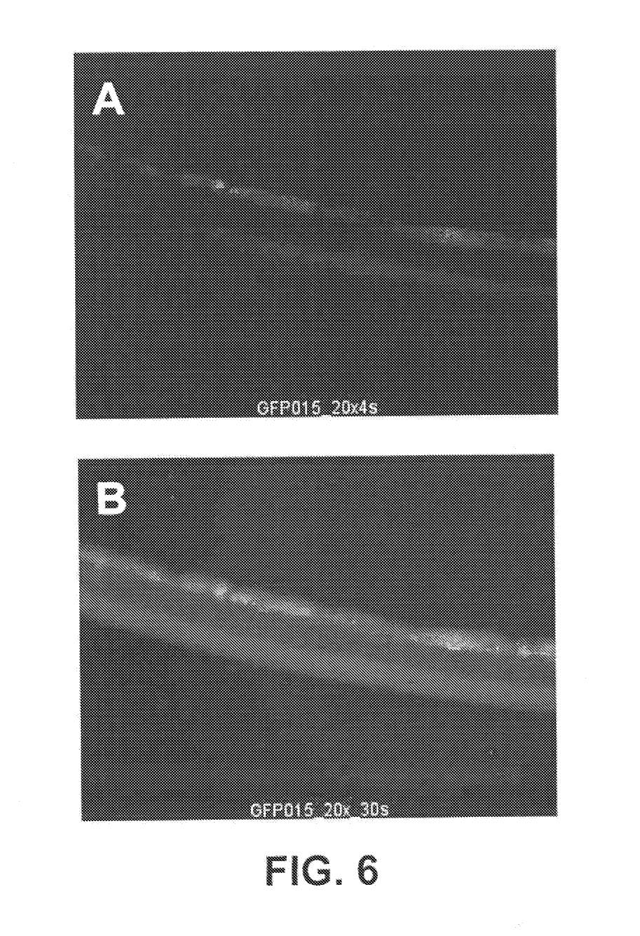

FIG. 6 (panels A and B) shows fluorescence images from the M0037-GFP samples. In most observations, fluorescence was much lower than that observed for the M0037-CI samples (Inclusion Bodies), and if images were captured in the same conditions, fluorescence was almost undetectable. However, there was some fluorescence signal present and when image capture time was lengthened to 30 seconds, some aggregates could be observed. Nevertheless, the distribution of the fluorescent signal was significantly more homogeneous than the signal observed in the samples incubated with GFP inclusion bodies. In some samples, a higher fluorescence intensity was detected, although faintly, with a 4 seconds of exposure time (results not shown).

### 4.4. M0037-CTRL Sample (Control)

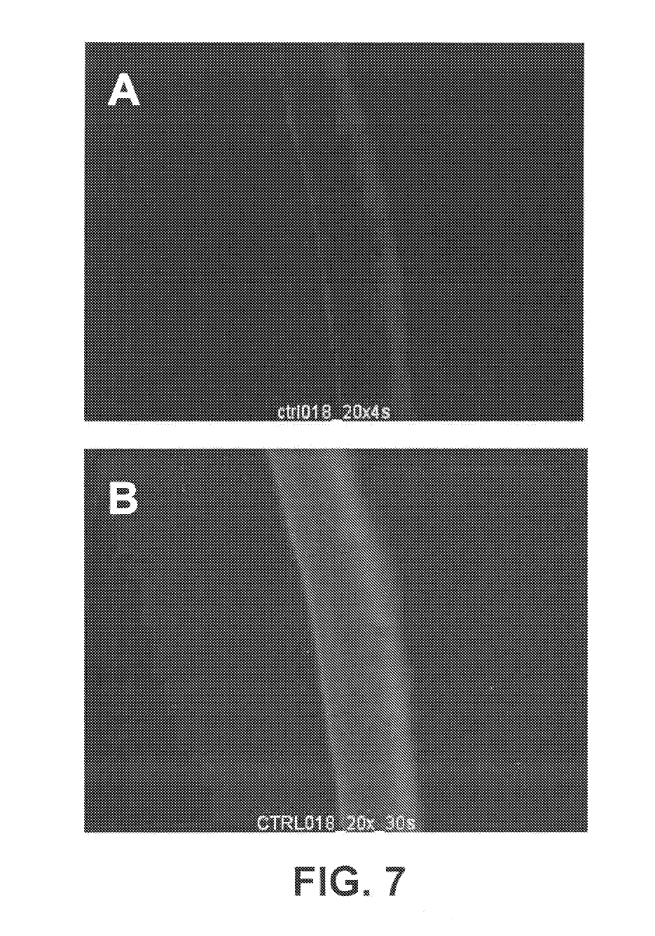

FIG. 7 shows fluorescence images corresponding to control sample M0037-CTRL. In all the observations, detected fluorescence was much lower than in the M00037-CI (GFP inclusion bodies) or M0037-GFP (soluble GFP) samples, and it was practically non-existent. Forcing the acquisition to an exposure time of 30 seconds, and opening the diaphragm to the maximum, it was possible to detected some faint fluorescence, which would be considered autofluorescence or background fluorescence since it was distributed homogeneously over the entire sample.

### 4.5 Images Obtained Using Confocal Microscopy

Three-dimensional reconstructions (Max projection) were performed using a series of optical sections obtained using a Leica SP2 confocal microscope (fluorescence images) and/or the images obtained with the transmitted light module in the same field in the case of phase contrast images. Images were obtained using 20 μm sections obtained according to the methods described above.

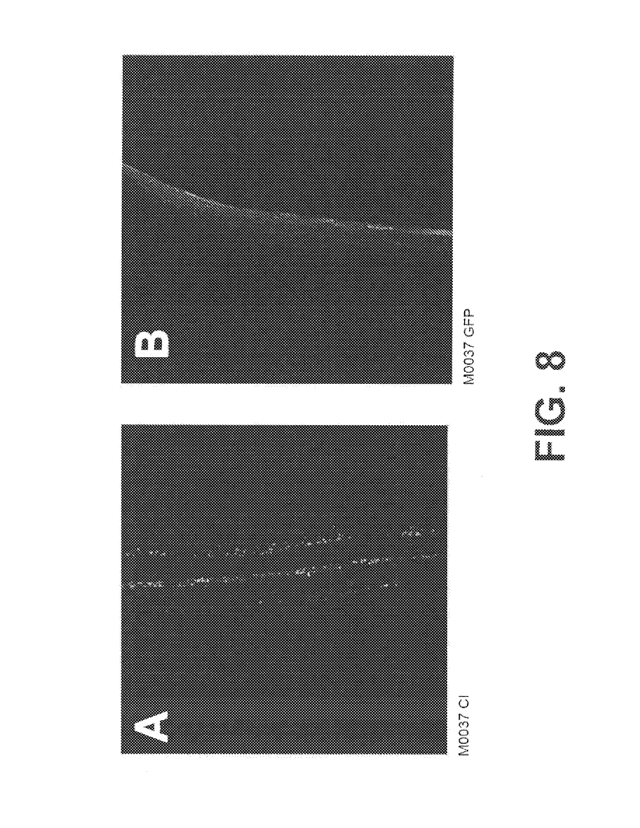

As shown in the FIG. 8 (20× magnification, same capture conditions for both series of images), there were clear differences in the fluorescence intensity and also in the shape and distribution of the signal from samples M0037-CI (inclusion bodies) (Panel A) and M00037-GFP (soluble GFP) (Panel B).

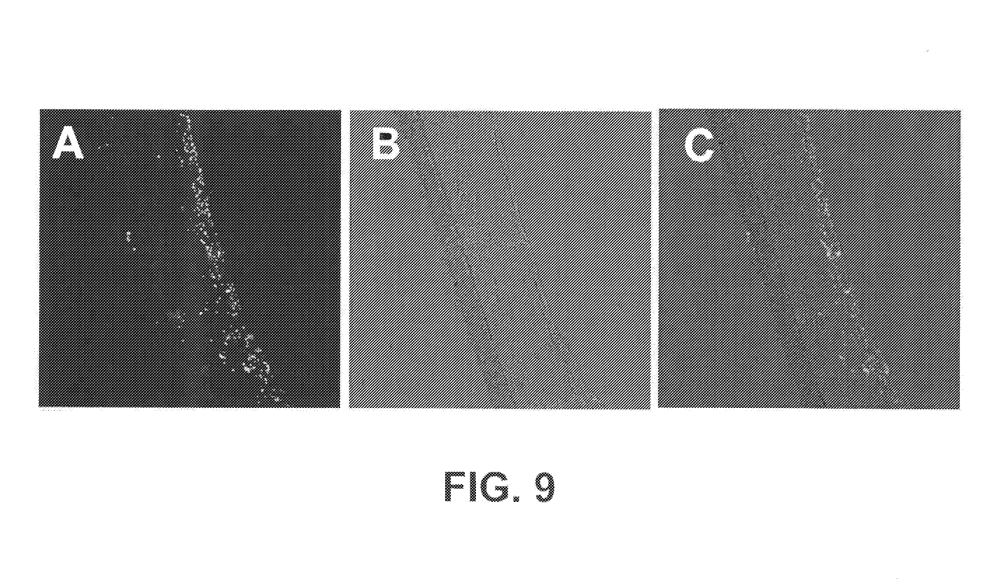

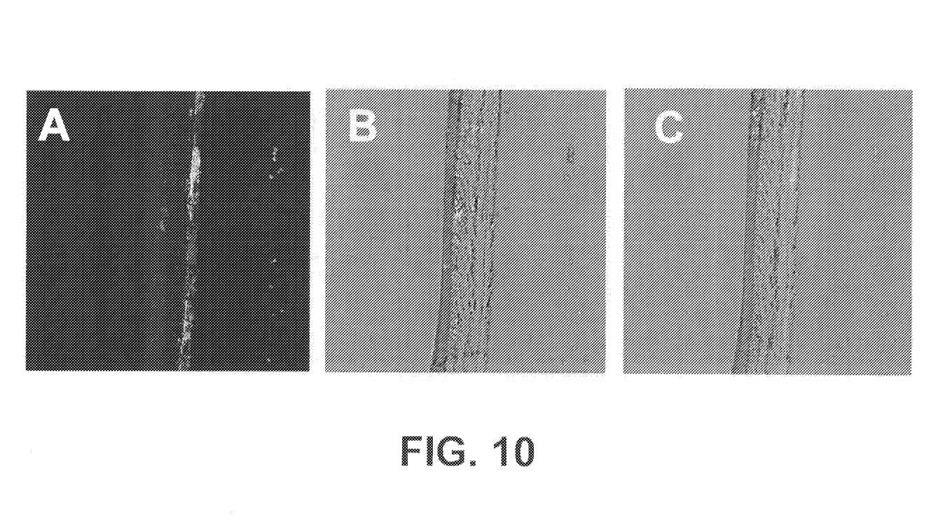

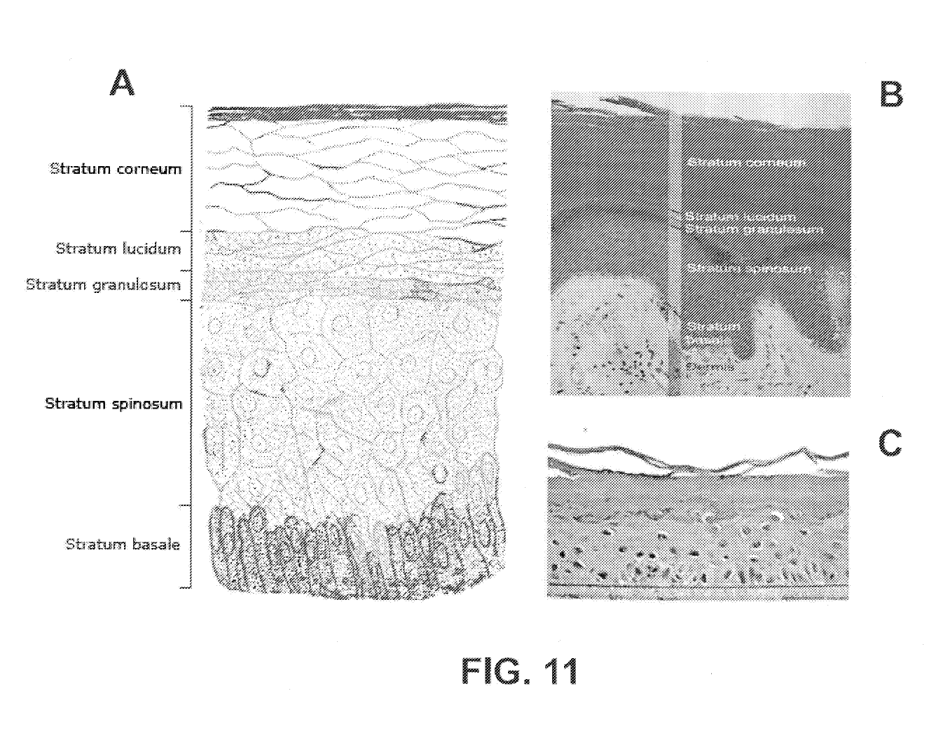

As shown in an example in FIG. 9, in most observations, the fluorescent signal from M0037-CI samples was detected as fluorescent aggregates located in the stratum corneum (SC), in the most external layer of the epidermis, and in many cases also in intermediate and deep areas of the epidermis. In contrast, as shown in an example presented in FIG. 10, in the majority of observations the fluorescence of the M0037-GFP samples was dispersed and located exclusively in the stratum corneum area.

These observations clearly indicated that inclusion bodies in particulate, non-solubilized form, were able to penetrate the epidermis. Furthermore, the penetration of the intact inclusion bodies was not superficial, with inclusion bodies being detected at intermediate and even at deep locations within the epidermis.

### Example 2

### Production and Characterization of GFP Inclusion Bodies

In general, inclusion bodies to be used for as cosmetic and/or therapeutic agents according to the disclosures in the instant application can be produced and characterized according to the methods disclosed in the instant example or methods known in the art. See, e.g., U.S. patent application Ser. No. 13/142,295 (published as U.S. Patent Publication No. US 2011-0268773), and U.S. patent application Ser. No. 13/319,772 (published as U.S. Patent Publication No. 2012-0148529), as well as all the references cited in those two U.S. patent applications which are herein incorporated by reference in their entireties.

Production of the Inclusion Bodies:

Inclusion bodies (IBs) were produced in Escherichia coli MC4100 strains (WT regarding protein folding and degradation, araD139 Δ(argF-lac) U169 rpsL150 relA1 flbB5301 deoC1 ptsF25 rbsR) and in a strain derived thereof, JGT20 (deficient in the main chaperone DnaK, dnak756 thr:Tn10), hereinafter DnaK strain. These strains were transformed with the expression vector pTVPIGFP (ApR) (Garcia-Fruitós et al. (2005) Microb. Cell. Fact. 4:27), encoding the green fluorescent protein (GFP) fused at the amino terminus to VP1, the pentamer-forming capsid protein of Foot and Mouth Disease Virus (FMDV) (Gonzalez-Montalban et al. (2007) Biochem. Biophys. Res. Commun. 355:637-642). This viral protein, being highly hydrophobic, directs the deposition of fusion proteins as inclusion bodies (Doglia et al. (2008) Biotechnol. J. 3:193-201). A similar construct, VP1LAC, encodes a previously described beta-galactosidase fusion (Garcia-Fruitós et al. (2005) Microb. Cell. Fact. 4: 27). The recombinant genes were expressed under the control of an isopropyl beta-D-1-thiogalactopyranoside (IPTG) inducible-trc promoter. The bacteria were cultured in Luria Bertani (LB) rich medium (Sigma-Aldrich, 28760 Madrid, Spain), supplemented with 100 μg/ml of ampicillin, and the recombinant gene expression was induced by adding 1 mM IPTG. Inclusion bodies are detectable after 1 hour of IPTG addition.

Purification of Inclusion Bodies:

Samples of 200 ml of bacterial cultures were centrifuged at 4° C. at 5.000 g for 5 minutes and resuspended in 50 ml of lysis buffer (50 mM TrisHCl pH 8.1, 100 mM NaCl and 1 mM EDTA). Ice jacketed samples were sonicated using a Braun LabsonicU probe sonicator (Braun Biotech International) for 25 to 40 minutes, at 40% of amplitude under 0.5 s cycles. Once sonicated, 28 μl of 100 mM phenylmethanesulphonylfluoride (PMSF) and 23 μl of lysozime were added to samples that were subsequently incubated at 37° C. under agitation for 45 min. After that, 40 μl of Nonidet P40 (NP-40) were added and the mixture is kept for 1 h at 4° C. under agitation. DNA was removed with 120 μl of 1 mg/ml DNase and 120 μl of 1 M Mg2SO4 for 45 min at 37° C. under agitation. Finally, samples were centrifuged at 4° C. at 15000 g for 15 min and the pellet, containing pure inclusion bodies, was washed with lysis buffer containing 0.5% Triton X-100 and stored at −20° C. until analysis.

Microscopic Analysis of Bacteria and Inclusion Bodies:

Samples were analyzed by using a Leica TSC SP2 AOBS confocal fluorescence microscope (Leica Microsystems Heidelberg GmbH, Manheim, Germany) after excitation at 488 nm, and images were recorded at emission wavelengths between 500 and 600 nm (63×(NA 1.4 oil) using a Plan Apochromat objective (zoom 8; 1,024 by 1,024 pixels). For the analysis of bacterial cells producing fluorescent inclusion bodies, samples taken 1, 2 or 3 h after IPTG induction were fixed with 0.2% formaldehyde in phosphate buffered saline (PBS) and stored at 4° C. until their use. Isolated inclusion bodies were resuspended in 20 ml of PBS.

Stability Analyses:

IBs obtained in DnaK-cells for 5 hours were diluted in PBS with 10 g/l bovine serum albumin (BSA) and 60 g/l sucrose, in the presence of gentamicin at 40 mg/l, penicillin at 100 U/ml and streptomycin at 10 μg/ml, and aliquots were incubated at different temperatures (37° C., 25° C., or 4° C.). At different times, samples were frozen at −80° C. until fluorescence determination.

Fluorescence was analyzed in a Cary Eclipse fluorescence spectrophotometer (Variant, Inc., Palo Alto, Calif.) by using an excitation wavelength of 450 nm and detecting the fluorescence emission at 510 nm. Results were referred to as the percentage of remaining activity or fluorescence with respect to control samples kept at −80° C., that were fully stable. Another set of samples was lyophilized in a Cryodos-80 lyophilizer, from Telstar (Terrassa, Spain) and stored at either 4° C. or 25° C. until analysis.

Confocal Laser Scanning Microscopy:

HeLa (cervical cancer cell line; ATCC: CCL-2™) and NIH3T3 (fibroblast cell line; ATCC: CRL-1658™) cell cultures were seeded at a density of 70% on glass plates (MatTek Corporation, Ashland, Mass., USA) 24 hours before adding VP1GFP inclusion bodies at different concentrations: 2 μM, 5 μM and 10 μM. Four hours after adding the inclusion bodies, the living cells were examined using a spectral confocal Leica TCS SP5 AOBS (Leica Microsystems, Mannheim, Germany) using a Plan Apochromat lens (63×, N.A. 1.4 oil). For nuclear and plasma membrane labeling, cells were incubated with 5 μg/ml of Hoechst 33342 and 5 μg/ml of CellMask (both from Molecular Probes, Inc., Eugene, Oreg., USA) respectively for 5 minutes at room temperature, and washed twice prior to confocal detection. Nuclei were excited with 405 nm diode laser beam, and detected at 414-461 nm (blue channel); plasma membrane was detected by exciting with the light of a 633 nm helium neon laser and fluorescence was detected at 656-789 nm (far red channel); finally, Argon laser 488-nm line was used for imaging VP GFP inclusion bodies (green channel, emission=500-537 nm).

### Example 3

### Production and Characterization of Hsp70 Inclusion Bodies

Inclusion bodies can be in some cases used both for cosmetic and for therapeutic purposed. Chaperones, such as Hsp70 or Hsp38 fall within this class of proteins. The human Hsp70 chaperone is a potent inhibitor of cell apoptosis (Gamido et al. (2003) Cell Cycle 2: 579-584), among other activities of therapeutic value (Calderwood et al. (2005) Eur. J. Immunol. 35: 2518-2527). Skin is the first barrier that protects the body against a great number of stressor agents. Cellular stress response involves an Hsp expression induction that has been reported to decrease with age, diminishing cell protection from environmental attacks. Increasing Hsp70 levels in the skin can be used as preventive cosmetics when skin is under hot and cold stress conditions, as photoprotection against UVB-induced cell death, in cells protection against dehydration, or to preventing damage caused by a vast number of stressors. See, e.g., Matsuda et al. (2010) J. Biol. Chem. 285: 5848-5858; Jonak et al (2006) Int. J. Cosmet. Sci. 28: 233-41; Laplante et al. (1998) J. Histochem. Cytochem. 46: 1291-301; Maytin (1992) J. Biol. Chem. 267: 23189-96; Bivik et al. (2007) Carcinogenesis 28: 537-44; Fargnoli et al. (1990) Proc. Natl. Acad. Sci. USA 87: 846-50; Garmyn et al. (2001) J. Invest. Dermatol. 117: 1290-5.

Production of Inclusion Bodies:

Following a protocol such as that described in Example 2, duly adapted, inclusion bodies are produced in strains of E. coli BL21 (DE3) transformed with a pReceiver-B01 commercial expression vector containing a N-His tag, a T7 promoter, and an ampicillin resistance gene (OmicsLink™ ORF Expression Ready Clone Catalog #EX-R0068-B1, GeneCopoeia, Rockville, Md.), expressing the human Hsp70 protein (Homo sapiens heat shock 70 kD protein 1B, HSPA 1B, NCBI Reference Number: NM005346; Genbank GI:167466172) with a Histidine-6 purification tag fused at the N-terminus. The bacterial cells are cultured in LB rich medium supplemented with 100 μg/ml of ampicillin, and the recombinant gene expression is induced by adding 1 mM IPTG. The inclusion bodies are detectable 1 hour after adding IPTG. The inclusion bodies formed by the aggregation of Hsp70 are purified following a procedure such as that described in Example 2.

Apoptosis Assay:

Cell apoptosis is determined, for example, using a fluorescent assay with Annexin V-FITC57 (e.g., an Annexin V-FITC Apoptosis Detection Kit (Roche)). Reconstituted epidermis samples are subjected to stressor agents (e.g., chemical products) or stressing environmental conditions known to cause apoptosis (e.g., cold, heat, UV radiation, dehydration) in the absence or presence of Hsp70 inclusion bodies. Epidermis samples can be treated with Hsp70 inclusion bodies previously, concurrently, or subsequently to the application of stressor agents or stressing environmental conditions. As controls, the same amounts of inclusion bodies are added to epidermis samples in the absence of the stressor agents or stressing environmental conditions, for the purpose of detecting putative deleterious effect of inclusion bodies on skin cells. After incubation for a period of time, skin cells are subjected to staining with Annexin V-FITC and propidium iodide, as recommended by the manufacturer, and fluorescence intensity levels are determined.

Results:

Incubation of epidermis samples with Hsp70 inclusion bodies will indicate whether human Hsp70 contained in non-solubilized inclusion bodies is able to perform its natural biological activities when administered topically in inclusion body form, and whether the inclusion bodies can significantly inhibit apoptotic events conducing to cell death. The results will indicate whether skin cells exposed to apoptosis-inducing conditions and treated previously, concurrently, or subsequently with Hsp70 inclusion bodies can maintain their viability. Furthermore, these observations show whether proteins produced in insoluble inclusion body form can be administered topically, whether these proteins are biologically active after topical administration, and whether the biological activity after topical administration has a therapeutic effect. Also, the results show whether inclusion bodies are nanoparticles with therapeutic value when administered topically, and whether, in addition, they are mechanically and functionally stable, and fully biocompatible.

### Example 4

### Catalase Inclusion Bodies

Reactive oxygen species (ROS) such as superoxide anion, hydroxyl radical, singlet oxygen, and hydrogen peroxide cause numerous deleterious effects on structural and functional (enzyme) proteins, lipid membranes, tissue polysaccharide, and genetic material (DNA). In skin, the molecules that are supposed to protect against these damages include specific enzymes such a superoxide dismutase (SOD), glutathione peroxidase (GPO), and catalase. Available enzymes from exogenous sources, such as catalase and SOD, usually are not easy to stabilize in cosmetic formulas.

Application of exogenous SOD for cosmetic uses has been described, for example, in Miyachi et al. (1987) J. Invest. Dermatol. 89:111-112; and, Filipe et al. (1997) Exp. Dermatol. 6:116-121. The use of similar enzymes obtained from thermophilic microorganisms as heat- and UV-stable cosmetic is also known in the art (see, e.g., Mas-Chamberlin et al. (2002) Cosmet. Toil 117:22-30; Lintner et al. (2002) IFSCC Magazine 5:195-200). See also, U.S. Pat. No. 4,129,644 (disclosing protecting skin and hair with cosmetic compositions containing superoxide dismutase), U.S. Pat. No. 5,145,644 (disclosing hydrogen peroxide destroying compositions and methods of making and using them), or EP1004289A2 (disclosing cosmetic and skin protective compositions comprising catalase).

Recently, catalase has also begun to be used in the aesthetics industry. Several mask treatments combine the enzyme with hydrogen peroxide on the face with the intent of increasing cellular oxygenation in the upper layers of the epidermis. Low levels of catalase also play a role in the graying process of human hair since hydrogen peroxide naturally produced by the body bleach the hair when catalase levels decline. Thus, catalase may be incorporated, for example, into cosmetic treatments for graying hair.

Production of Catalase Inclusion Bodies:

Culture samples of 20 ml were harvested by centrifugation at 5.000 g at 4° C. for 5 minutes, resuspended in lysis buffer (50 mM TrisHCl pH 8.1, 100 mM NaCl and 1 mM EDTA) and frozen at −80° C. After thawing, 100 μl, 100 mM of phenylmethanesulphonylfluoride (PMSF) (or other protease inhibitor) and 400 μl of 50 mg/mL lysozime were added and samples were incubated at 37° C. for 2 hours. After the incubation, 100 μl of the same lysis buffer containing 0.5% Triton X-100 were added and incubated at room temperature for 1 hour. Then, samples were disrupted using sonication or another disruption method, such as high pressure homogenization. After that, 5 μl of Nonidet P40 (NP-40) were added, and samples were incubated at 4° C. for 1 hour. Then, DNA was removed with 15 μl of 1 mg/ml DNase and 15 μl 1M MgSO4 for 45 minutes at 37° C. Finally, samples were centrifuged at 4° C. at 15000×g for 15 minutes, and the pellet containing pure catalase inclusion bodies was washed once with 1 ml of lysis buffer containing 0.5% Triton X-100. After a final centrifugation at 15000×g for 15 minutes at 4° C., pellets were stored at −80° C. until analysis. All incubations were done under agitation. The volumes and incubation times used in this protocol were scaled up when using higher amounts of sample.

In preliminary experiments to determine whether catalase in inclusion body form was pharmacologically active, catalase inclusion bodies were administered to an in vitro neuron model system. Catalase inclusion bodies were found to be enzymatically active and to have a neuroprotective effect in the model system (results not shown).

Results:

Incubation of epidermis samples with catalase inclusion bodies will indicate whether catalase contained in non-solubilized inclusion bodies is able to perform its natural biological activities when administered topically in inclusion body form, and whether the inclusion bodies can significantly protect the cells from oxidative damage by reactive oxygen species (ROS). The results will indicate whether skin cells exposed to reactive oxygen species and treated previously, concurrently, or subsequently with catalase inclusion bodies can maintain their viability.

### Example 5

### Interleukin-10 (IL-10) Inclusion Bodies

Interleukin-10 (IL-10 or IL10), also known as human cytokine synthesis inhibitory factor (CSIF), is an anti-inflammatory cytokine. One role of IL-10 may be to prevent severe damage to the skin by reducing the risk of necrosis by ongoing inflammatory processes. See, e.g., Grimbaldeston et al. (2007) Nature Immnology 8:1095-1104 (disclosing that mas cell-derived interleukin-10 limits skin pathology in contact dermatitis, e.g., in response to poison ivy or poison oak, and in chronic irradiation with ultraviolet B). Accordingly, IL-10 may be incorporated in topical compositions to reduce skin inflammation.

Production of Inclusion Bodies:

Culture samples of 20 ml are harvested by centrifugation at 5.000×g at 4° C. for 5 minutes, resuspended in lysis buffer (50 mM TrisHCl pH 8.1, 100 mM NaCl and 1 mM EDTA) and frozen at −80° C. After thawing, 100 μl, 100 mM of phenylmethanesulphonylfluoride (PMSF) (or other protease inhibitor) and 400 μl of 50 mg/mL lysozime are added and samples are incubated at 37° C. for 2 hours. After the incubation, 100 μl of the same lysis buffer containing 0.5% Triton X-100 is added and incubated at room temperature for 1 hour. Then, samples are disrupted using sonication or another disruption method, such as high pressure homogenization. After that, 5 μl of Nonidet P40 (NP-40) are added, and samples are incubated at 4° C. for 1 hour. Then, DNA is removed with 15 μl of 1 mg/ml DNase and 15 μl 1 M MgSO4 for 45 min at 37° C. Finally, samples are centrifuged at 4° C. at 15000×g for 15 minutes, and the pellet containing pure IL-10 inclusion bodies is washed once with 1 ml of lysis buffer containing 0.5% Triton X-100. After a final centrifugation at 15000×g for 15 minutes at 4° C., pellets are stored at −80° C. until analysis. All incubations are done under agitation. The volumes and incubation times used in this protocol are scaled up when using higher amounts of sample.

Results:

Incubation of epidermis samples with IL-10 inclusion bodies will indicate whether IL-10 contained in non-solubilized inclusion bodies is able to perform its natural biological activities when administered topically in inclusion body form, and whether the inclusion bodies can significantly prevent severe damage to the skin by reducing the risk of necrosis by ongoing inflammatory processes, can limit skin pathology, or can reduce skin inflammation. The results will indicate whether skin cells exposed to inflammation-causing stimuli and treated previously, concurrently, or subsequently with IL-10 inclusion bodies can maintain their viability.

### Example 6

### Use of Interleukin-10 (IL-10) Inclusion Bodies for the Treatment of Psoriasis

IL-10 has been used for the treatment of psoriasis in clinical trials, in particular through subcutaneous administration to patients. However, given the high cost associated with the production of the protein, amounts required, and its chronic administration, the use of IL-10 for the treatment of psoriasis is economically impractical. Topical administration of IL-10 would significantly reduce the cost of treatment with IL-10.

To test the feasibility of using IL-10 inclusion bodies to treat psoriasis, IL-10 inclusion bodies were used in an ex vivo model system comprising cultured biopsies (explants) of human skin with psoriasis. This model system can maintain the histological, cellular, and genetic characteristics of human skin during several days in culture. Thus, this model system can be used to evaluate the effects of different stimuli/inhibitors on skin diseases or conditions such as psoriasis and atopic dermatitis. In addition, this technique can be combined with quantitative RT-PCR to evaluate the pharmacological activity of the tested agents.

Gene expression studies using psoriatic and healthy skin allowed the identification and validation of genes overexpressed in the psoriatic lesion and to correlate their levels with the clinical efficacy of the treatments. The identification of genes associated with the mechanism of action of IL-10 and/or related with psoriatic pathology allowed the evaluation of the activity of IL-10 inclusion bodies applied ex vivo to the psoriatic lesions. Biopsies were obtained from patients, and in each biopsy the expression levels of 10 genes were determined via RT-PCT.

IL-10 inclusion bodies were applied to psoriatic explants from patients. Each explant was subdivided into four portions, which were respectively used to conduct 4 different assays: (i) two tests, each one with a different concentration of IL-10 inclusion bodies applied topically to the skin sample, (ii) a control test in which GFP inclusion bodies (GFP-VP1) were applied to the skin samples, and (iii) a negative control (without inclusion bodies).

In psoriatic explants treated with IL-10 inclusion bodies a significant decrease in inflammation was observed (decreased levels of IFN-gamma, IL-8, K16, TNF-alpha, and IL-17A were observed). In explants treated with GFP inclusion bodies, a slight increase in inflammation was observed with respect to the control samples (see TABLE 3).

TABLE 3
Effect of IL-10 inclusion bodies on Inflammation

| | | | GFP Inclusion body | | IL-10 inclusion body | |
|---|---|---|---|---|---|---|
| | CONTROL | | (100 ng/ml) | | (100 ng/ml) | |
| Gene | Ct mean | Normalized | Ct mean | Normalized | Ct mean | Normalized |
| B-defensin 4 | 32.50 | 3761.92 | 31.33 | 4933.45 | 32.25 | 3798.27 |
| ICAM-1 | 35.18 | 779.75 | 33.84 | 1127.96 | 34.64 | 933.46 |
| IFN-gamma | 29.42 | 64.35 | 36.67 | 214.02 | Undetermined | 0 |
| IL-23p19 | 36.26 | 412.76 | 36.10 | 299.55 | 37.05 | 226.08 |
| IL-8 | 26.65 | 117295.35 | 24.66 | 248036.47 | 29.53 | 18732.89 |
| IP-10 | 35.54 | 632.28 | 35.08 | 545.28 | 34.41 | 1065.43 |
| K16 | 32.49 | 3790.52 | 31.14 | 5509.05 | 33.07 | 2341.25 |
| TNF | 34.15 | 1428.18 | 33.87 | 1108.27 | 36.55 | 303.26 |
| IL17A | 36.70 | 328.55 | 36.04 | 309 | 36.68 | 281.21 |
| GADPH | 30.84 | | 30.13 | | 30.60 | |

Normalization: For each condition, 1.8e(Ct GADPH-Ct problem gene) × 10,000 (see Chan et al, J. Exp. Med. 203: 2577-2587 (2006).

These results showed that the administration of IL-10, an agent which can be used therapeutically (to reduce inflammation) or as a cosmetic agent (to reduce visual effects of inflammation, such as redness and/or swelling), in inclusion body form was able to reduce inflammation in an ex vivo human psoriasis model system.

The observation that GFP-VP1 inclusion bodies, used as a control/placebo, caused a slight inflammatory effect suggested that the use of E. coli-produced inclusion bodies may have a slight immunogenic effect. Accordingly, an alternative inclusion body production system was developed used probiotic bacterial strains, which are organisms recognized as GRAS (results not shown).

### Example 7

### EGF, KGF and VEGF Inclusion Bodies

Summary:

Genes encoding EGF, VEGF and KGF were cloned into two P170 vectors (Bioneer A/S, Horsholm, Denmak). The sequences of the cloned constructs were confirmed by DNA sequencing. Three different L. lactis strains were transformed, generating in total 18 different strains. Flask expression experiments were performed for strains expressing KGF, EGF, or VEGF. Fast-prep lysates were prepared, yielding soluble and insoluble fractions. Western blot analyses were conducted using either the soluble or the insoluble fractions. Western blot analysis showed expression of KGF mainly in the insoluble fraction (both in the high copy number plasmid and clpP strain). VEGF expression was detected both in soluble and insoluble fractions, although there was a different band pattern in each fraction.

Construction of Expression Plasmids in E. coli

Codon optimized genes encoding human EGF (55 amino acids long), KGF (165 amino acids long) and VEGF (208 amino acids long) were synthesized. Two expression vectors were used for gene cloning in E. coli (i) pAMJ398, a medium-copy number plasmid (MCN) for intracellular production, and (ii) pAMJ328, a high-copy number plasmid (HCN) for intracellular production. The genes were cloned into expression vectors via NcoI and SalII restriction sites of pAMJ328, and via BspHI and SAlI restriction sites of pAMJ398. Expression plasmids were established in E. coli DH10B (Research Master Cell Banks/rMCB was stored at −80° C.): UP1406: pAMJ328:EGF; UP1407: pAMJ328:VEGF; UP1408: pAMJ328:KGF; UP1409: pAMJ398:EGF; UP1410: pAMJ398:VEGF; and, UP1411: pAMJ398:KGF.

Transformation of Lactococcus lactis Strains:

Plasmid DNA was purified from E. coli strains. Plasmid DNA was used for restriction enzyme mapping, DNA sequencing of the cloning junctions and transformation of three L. lactis strains based on MG1363 (Bioneer A/S, Horshoim, Denmak); wt, htrA−, clpP−. All plasmid constructions were confirmed and validated by restriction enzyme mapping and DNA sequencing. rMCBs were established in glycerol (stored at −80° C.) after growth in M17G5-erm.

TABLE 4
Transformed L. lactis strains

| | Strain | | | | | |
|---|---|---|---|---|---|---|
| | Wt | | htrA | | clpP | |
| Gene/vector | pAMJ328 | pAMJ398 | pAMJ328 | pAMJ398 | pAMJ328 | pAMJ398 |
| EGF | UP1420 | UP1423 | UP1426 | UP1429 | UP1432 | UP1435 |
| VEGF | UP1421 | UP1424 | UP1427 | UP1430 | UP1433 | UP1436 |
| KGF | UP1422 | UP1425 | UP1428 | UP1431 | UP1434 | UP1437 |

L. lactis Flask Experiments:

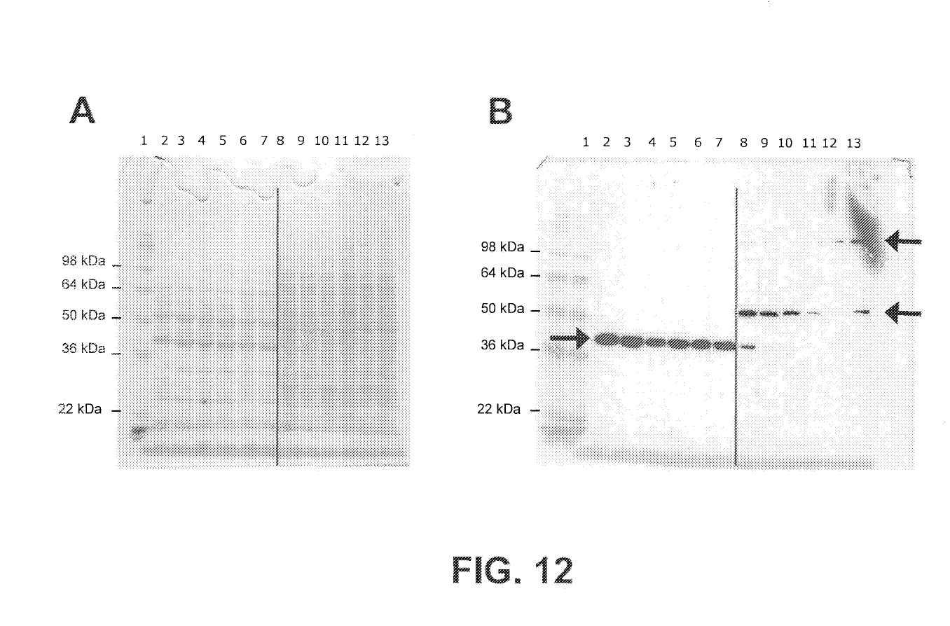

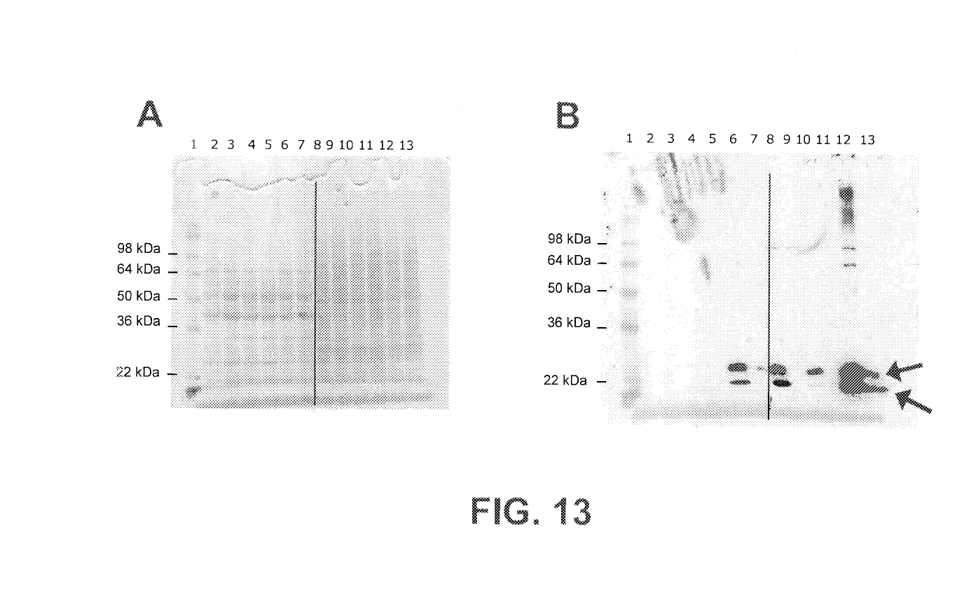

The 18 developed L. lactis strains (TABLE 4) were grown over night in rich medium (1.5×M17) supplemented with 1% glucose and 1 μg/mL erythromycin. pH and OD₆₀₀ after growth overnight were measured to ensure that induction of the P170 promoter took place. The pH was around 5.0-5.5 and OD₆₀₀ around 4-5 as expected. 10 ml of the over night cultures were harvested and the cell pellets were stored at −20° C. The cell pellet was washed in 1 ml PSB and the cell material was divided into two tubes each containing 500 μl cell material (˜5 ml culture). One pellet was used for fast-prep and the other was stored at −20° C. Soluble and insoluble fractions were prepared using a modified protocol for fast-prep. Soluble fraction and insoluble fractions were analyzed by SDS-PAGE followed by Coomassie staining and Western blot analysis (see FIG. 12 and FIG. 13).

L. lactis Flask Experiment (VEGF):

L. lactis cells containing expression vectors with VEGF were grown over night at 30° C. in 1.5×M17G10 medium with erythromycin. 10 μL of the soluble intracellular fraction and 10 μL of the insoluble intracellular fraction were loaded on an SDS-gel 12% Tris-glycin, Coomassie stained (FIG. 12A) and Western blotted (FIG. 12B).

No obvious protein corresponding to the molecular weight of VEGF was seen by Coomassie staining, neither soluble nor insoluble (FIG. 12A). However, a clear signal was seen by Western blotting in the soluble fractions (in all strains, lanes 2-7, indicated by arrow on left side of blot) (FIG. 12B). The Mw was around 40-42 kDa, which was higher than the predicted band size (23 KDa). The presence of VEGF bands with this Mw is also described in the product datasheet from Abcam (VEGF antibody supplier) when analyzing expression of VEGF in human cell lines. Bands of ca. 50 KDa and >100 kDa were detected in the insoluble fractions in most strains (lanes 8-13, indicated by arrows on right side of blot), indicating that VEGF was present in inclusion bodies.

L. lactis Flask Experiment (KGF):

L. lactis cells containing expression vectors with KGF were grown over night at 30° C. in 1.5×M17G10 medium with erythromycin. 10 μL of the soluble intracellular fraction and 10 μL of the insoluble intracellular fraction were loaded on an SDS-gel 12% Tris-glycin, Coomassie stained (FIG. 13A) and Western blotted (FIG. 13B).

No obvious protein corresponding to the molecular weight of KGF was seen by Coomassie staining, neither soluble nor insoluble (FIG. 13A). However, clear signals were seen by Western blotting in the soluble fractions in the clpP strain (double band in lanes 6-7, arrows on the right side of the blot) (FIG. 13B). The M_{W} was around 22 KDa, which is close to the predicted band size for KGF (18 KDa). The same band pattern was seen in the insoluble fractions. The most intense band pattern was seen in the clpP strain containing the high-copy number (HCN) plasmid (lane 12, strain UP1437), indicating that KGF was present in inclusion bodies.

L. lactis Flask Experiment (EGF):

L. lactis cells containing expression vectors with EGF were grown over night at 30° C. in 1.5×MI7G10 medium with erythromycin. 10 μL of the soluble intracellular fraction and 10 μL of the insoluble intracellular fraction are loaded on an SDS-gel 12% Tris-glycin, Coomassie stained and Western blotted. These experiments will indicate whether EGF expressed in the disclosed strains is present in inclusion bodies.

Incubation of epidermis samples with EGF and/or KGF and VEGF inclusion bodies (or compositions comprising also other protein or non-protein therapeutic agents, excipients, etc.) will indicate whether these growth factors, alone or in combination, contained in non-solubilized inclusion bodies are able to perform their natural biological activities when administered topically in inclusion body form (i.e., administered to skin or other epithelial tissues), and whether the inclusion bodies can significantly prevent, ameliorate, or treat intrinsic and/or extrinsic skin damage (e.g., due to aging or exposure to environmental factors) or pathological conditions (e.g., skin diseases or conditions). Accordingly, the experimental data will indicate the ability of these inclusion bodies to function effectively as cosmetics and/or therapeutic agents. The results will also indicate whether skin cells (e.g., in artificial model systems, ex vivo models, or the skin of subjects) exposed to skin-damaging conditions and treated previously, concurrently, or subsequently with compositions comprising EGF and/or KGF and VEGF inclusion bodies inclusion bodies can maintain their viability.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1-51. (canceled)

52. A method for treating a skin condition in a subject in need thereof comprising topically applying a cosmetically or therapeutically effective amount of a cosmetic or therapeutic agent composition comprising at least one cosmetic or therapeutic agent in inclusion body form, and a dermatologically or pharmaceutically acceptable carrier to the skin of the subject so as to improve the skin condition of the subject.

53. The method according to claim 52, wherein the inclusion body is insoluble, not solubilized, partially solubilized, or in particulate form.

54. The method according to claim 53, wherein the particulate form has a particle size between 20 and 1500 nm.

55. The method according to claim 53, wherein the particulate form is in hydrated amorphous form.

56. The method according to claim 52, wherein the inclusion body is internalized by a target cell.

57. The method according to claim 56, wherein the target cell is an epidermal cell, a non-epidermal cell, a neuron, a muscle cell, or an adipocyte.

58. The method according to claim 52, wherein the inclusion body can penetrate at least one skin layer.

59. The method according to claim 58, wherein the one skin layer is selected from the group consisting of the cornified layer (stratum corneum), the translucent layer (stratum lucidum), the granular layer (stratum granulosum), the spinous layer (stratum spinosum), the basal/germinal layer (stratum basale/germinativum), and a combination thereof.

60. The method according to claim 52, wherein the cosmetic agent or therapeutic agent comprises a polypeptide.

61. The method according to claim 60, wherein the polypeptide is biologically active.

62. The method according to claim 60, wherein the polypeptide is a prodrug.

63. The method according to claim 60, wherein the polypeptide is a recombinant polypeptide or a biologically active fragment thereof, a natural polypeptide or a biologically active fragment thereof, or a chemically synthesized polypeptide.

64. The method according to claim 60, wherein the polypeptide is a chimeric protein, a fusion protein, or a protein conjugate.

65. The method according to claim 61, wherein the recombinant polypeptide or fragment thereof is expressed in bacteria, yeast, insect, or mammalian cells.

66. The method according to claim 60, wherein the polypeptide is genetically fused or conjugated to an inclusion-body inducing polypeptide.

67. The method according to claim 60, wherein the polypeptide comprises, consists, or consists essentially of IL-10 and/or EGF and/or KGF and/or VEGF, and/or fragments, variants, or derivatives thereof.

68. The method according to claim 52, wherein said skin condition is selected from psoriasis, cellulite, acne, skin aging, skin wrinkles, hyperpigmentation, keratosis, skin blemish, dandruff, warts, photodamaged skin, chronic dermatoses, dermatitis, dryness, ichthyosis, viral infections, fungal infections, bacterial skin infections, athlete's foot, canker sore, carbuncle, candidiasis, bacterial vaginitis, vaginosis, cold sores, dandruff, eczema, erythrasma, erysipelas, erythema multiforme, furuncle, and impetigo.

69. The method according to claim 52, wherein cosmetic or therapeutic agent composition is a solution, a gel, a cream, a lotion, an ointment, an emulsion, a suspension, an aerosol, an aerosol foam, a liniment, a tincture, a salve, a poultice, or a dry power.

70. A method of delivering a cosmetic or therapeutic agent across the skin barrier comprising applying to the skin of a subject a cosmetic or therapeutic composition comprising at least one cosmetic or therapeutic agent in inclusion body form, wherein the at least one cosmetic or therapeutic agent crosses the skin barrier in inclusion body form.

71. A method of stimulating tissue regeneration, comprising applying to the skin of a subject at least one cosmetic agent or therapeutic agent in inclusion body form, wherein the inclusion body penetrates the skin barrier and reaches said tissue and stimulates its regeneration
